(19) United States
(12) Patent Application Publication
(10) Pub. No.: US 2003/0186254 A1
(43) Pub. Date: Oct. 2, 2003

(54) Regulation of HIV-Tat and Nef by PAK4 kinase and its binding partners and methods of identifying modulators thereof

(75) Inventors: Michael B. Melnick, Belmont, MA (US); Albrecht Moritz, Salem, MA (US); Michael J. Comb, Manchester, MA (US)

Correspondence Address: James Gregory Cullem, Esq., Intellectual Property Counsel, CELL SIGNALING TECHNOLOGY, INC., 166B Cummings Center, Beverly, MA, 01915, US

(73) Assignee: CELL SIGNALING TECHNOLOGY, INC.

(21) Appl. No.: 10/134,102

(22) Filed: Apr. 29, 2002

Related U.S. Application Data
(63) Continuation-in-part of application No. 09/750,457, filed on Dec. 28, 2000, now abandoned.
(60) Provisional application No. 60/173,939, filed on Dec. 30, 1999.

(51) Int. Cl. C12Q 1/70; C12Q 1/68; C12P 19/34; C12N 7/00; C12N 7/01; C12N 5/06; C12N 5/16

(52) U.S. Cl. 435/6; 435/91.1; 435/235.1; 435/345

(57) ABSTRACT

The present invention discloses complexes of cellular signaling proteins that interact in vivo with the HIV-encoded auxiliary proteins Nef and Tat to modulate their activity. This complex includes the novel serine/threonine kinase PAK4 and the novel guanine nucleotide exchange factor Cdc42-GEF, which synergize to stimulate Tat transcriptional activity, and the acetyl-transferase Tip60 which modifies Nef. These cellular partners of the HIV auxiliary proteins represent novel targets for HIV therapeutics. The invention provides isolated DNA and vectors encoding PAK4 and Cdc42-GEF, and methods of producing recombinant forms of these proteins. The invention also provides methods for identifying compounds that modulate the activity of HIV-Tat, HIV-Nef or Tip60, and methods for modulating the activity of these enzymes.

### RELATED APPLICATIONS

This application is a continuation-in-part of, and claims priority to, U.S. Ser. No. 09/750,457, filed Dec. 28, 2000, presently pending, and U.S. Ser. No. 60/173,939, filed Dec. 30, 1999, now abandoned, the disclosures of which are incorporated by reference herein.

### FIELD OF THE INVENTION

The invention relates generally to protein-protein interactions, and more particularly to interactions involving viral transcriptional enzymes.

### BACKGROUND OF THE INVENTION

The handful of proteins encoded by the genome of the Human Immunodeficiency Virus (HIV) have been the object of intense scrutiny for clues as to the mechanism of the disease AIDS and as targets for potential AIDS therapeutics. Today's most effective drugs in the treatment of AIDS were designed either as inhibitors of the HIV-encoded DNA polymerase or the HIV protease. However there are a number of other, smaller, HIV-encoded proteins, so-called auxiliary proteins, whose function still remains unclear (Cullen (1998) Cell 93:685-692). These proteins and their cellular effector proteins represent additional potential targets for the development of future AIDS therapeutics.

One of the most poorly understood of these auxiliary proteins is HIV-Nef (reviewed in Marsh (1999) Arch Biochem Biophys. 365(2):192-198). Among several putative cellular Nef-binding proteins that may hold clues as to its in vivo function is a cellular serine/threonine kinase activity (Nef-associated kinase or NAK, reviewed in Trono and Wang (1997) Chemistry and Biology 4:13-15). Nef is thought to activate NAK which may help mediate the role Nef plays in HIV replication (Lu et al. (1996) Current Biology 6(12):1677-1684). NAK is believed to be a member of the PAK or p21-activated kinase family by virtue of its cross-reactivity with anti-PAK antibodies and its regulation by Cdc42 and Rac.

As presently disclosed, NAK has now been identified as a novel member of the PAK family presently named “PAK4” (this protein has since been independently identified by Abo et al. (1998) EMBO Journal 17(22):6527-6540). The PAK family of kinases represents a growing group of kinases related in the sequence of their kinase domains and in their binding and regulation by Rho-family small G proteins such as Rac and Cdc42 (reviewed in Manser and Lim (1999) Progress in Molecular Subcellular Biology 22:115-133; Knaus and Bokoch (1998) Intl. J. Biochem. Cell Biol. 30:857-862). The three PAK isoforms characterized in mammals to date have been designated Pak1 (also known as alpha-Pak), Pak2 (also known as gamma-Pak), and Pak3 (beta-Pak). Pak1 and Pak3 are expressed primarily in the brain, while Pak2 tissue expression is ubiquitous. These Pak family members have been characterized primarily in terms of their role in regulation of the cytoskeleton and morphogenesis, and as upstream activators of the JNK and p38 MAPK pathways. Recently NAK was reported to be identical to Pak2 (Renkema et al. (1999) Current Biology 9:1407-1410); however it was not possible to reproduce the interaction between Nef and Pak2 described in that report and instead Pak4 was identified as the Nef-associated kinase.

Pak1 and Pak3 have previously been shown to bind proteins with guanine nucleotide exchange factor activity, members of the so-called Cool/Pix family (reviewed in Bagrodia and Cerione (1999) Trends in Cell Biology 9:350-355). These enzymes presumably facilitate Pak activation by charging Rho-family G proteins with GTP which then bind and activate the associated Pak. In this application we characterize a novel guanine nucleotide exchange factor that is the corresponding binding partner of Pak4 and seems to show exchange factor activity specific for cdc42 (this protein has since been identified by Fukuhara et al. (1999) Journal of Biological Chemistry 274(9):5868-5879).

Besides Nef another essential HIV auxiliary protein, namely the unique HIV-encoded transcription factor Tat, has also been found to associate with cytoplasmic serine/threonine kinase activity (reviewed in Karn (1999) Journal of Molecular Biology 293:235-254 and Taube et al. (1999) Virology 264:245-253). Tat recruits two cyclin-dependent-kinase-containing complexes (Cdk9/P-TEFb and Cdk7/TFIIH) to the HIV-LTR where by phosphorylating RNA polymerase 11 they contribute to transcriptional elongation. Until now these are the only Tat-associated cellular kinase activities to have been characterized.

HIV-Tat also binds a cellular protein called Tip60 or 60 kD Tat-interacting protein (Kamine et al. (1996) Virology 216:357-366). The sequence (SEQ ID NO:9 and SEQ ID NO:10) of this protein shows homology to known protein acetyl transferase enzymes, and indeed Tip60 has been shown to have acetyltransferase activity as assayed in vitro on histones (Kimura and Horikoshi (1998) Genes to Cells 3:789-800). A good in vivo substrate for this enzyme has until now not been identified however.

Accordingly, there remains a need for the elucidation of protein-protein binding interactions that regulate the activity of HIV-Tat and/or NEF. The identification of such interactions would be useful, for example, in screening for compounds that modulate the activity of Tat and/or NEF, and would enable the modulation of the activity of these proteins by modulating their protein-protein complex formation.

### SUMMARY OF THE INVENTION

In accordance with the present invention, it has now been demonstrated that HIV-Tat and HIV-Nef bind a complex of cellular signaling proteins including the serine/threonine kinase PAK4, the guanyl nucleotide exchange factor cdc42-GEF and the acetyl-transferase Tip60. Using an HIV-LTR transcriptional reporter assay we show that PAK4 and Cdc42/GEF synergize to activate Tat transcriptional activity, while Nef and Tip60 synergize to inactivate Tat. As effectors or modulators of the activity of the HIV auxiliary proteins, the cellular proteins PAK4, cdc42-GEF and Tip60 represent novel targets for the development of therapeutics directed against HIV. We also show that HIV-Nef is an in vivo substrate of Tip60 acetyl-transferase activity, providing a novel assay for the development of drugs that modulate Tip60 activity.

The invention provides, in part, isolated DNA sequences encoding a novel PAK4 serine/threonine kinase, and a vector for expressing Cdc42-specific GEF (guanyl-nucleotide exchange factor), the vector comprising a DNA sequence encoding the same. Also provided are a method for producing PAK4 or Cdc42-GEF protein by the steps of transfecting a cell with a vector comprising a DNA sequence encoding PAK4 or Cdc42-GEF, and culturing the cell in order to express the desired vector.

The invention also provides methods for modulating the transcriptional activity of HIV Tat protein by modulating the formation of a complex between Tat and at least one modulator complex comprising PAK4 and one or more binding proteins, and methods for identifying a compound that inhibits the transcriptional activity of HIV-Tat by determining whether the compound disrupts the formation of a complex including PAK4 and Cdc42-GEF, or whether the compound enhances the formation of a complex including PAK4, HIV-NEF, and Tip60. Also provided are methods of inhibiting the transcriptional activity of HIV-Tat using a compound that decreases or increases activity or expression of a HIV-Tat complex binding protein. Methods for modulating the activity of HIV-NEF, identifying a compound that modulates HIV-NEF acetylation by Tip60, and identifying a compound that modulates Tip60 acetyl-transferase activity are also provided.

### BRIEF DESCRIPTION OF THE DRAWINGS

FIG. 1 illustrates human multiple tissue Northern blot (Clontech) probed with PAK4 cDNA, revealing a ubiquitous 2.9 kb mRNA (liver signal visible on long exposure).

FIG. 2A is a schematic of the structure of the PAK4 protein.

FIG. 2B is a schematic of the structure of the GEF protein.

FIG. 3 illustrates the epitope-tagged eukaryotic expression constructs used in co-immunoprecipitation experiments.

FIG. 4A illustrates the co-precipitation of GST-PAK4 detected by anti-GST Western blot, showing that PAK4 binds GEF, Tat and Tip60 and Nef in vivo.

FIG. 4B illustrates the co-precipitation of FLAG-GEF detected by anti-FLAG Western blot, showing that GEF specifically binds PAK4 but not PAK2 in vivo. GEF interacts with both the kinase domain and the amino-terminal regulatory domain of PAK4.

FIG. 5A illustrates co-precipitation of FLAG-Tat detected by anti-FLAG Western blot, showing that HIV-Tat specifically binds PAK4 not PAK2 in vivo. Tat interacts with the amino-terminal regulatory domain, not the kinase domain, of PAK4.

FIG. 5B illustrates the co-precipitation of FLAG-Nef detected by anti-FLAG Western blot. While an interaction between HIV-Nef and full-length PAK4 was not seen, a specific interaction between Nef and the amino-terminal regulatory domain of PAK4 (but not PAK2) was observed. The Nef-PAK4 interaction may occur in vivo at a point in the regulation of PAK4 activity when this binding domain is exposed.

FIG. 6 illustrates co-precipitation of FLAG-tagged GEF, Nef and Tat detected by anti-FLAG Western blot, indicating the presence of distinct binding sites on PAK4 for Tat and Nef. Nef binds at the extreme amino terminus of PAK4, between amino acids 1 and 91 where the Cdc42-binding motif is also found, while Tat binds PAK4 between the Cdc42-binding motif and the kinase domain (between amino acids 93 and 290).

FIG. 7A illustrates the co-precipitation of Cdc42 detected by anti-Cdc42 Western blot, showing that Cdc42 binds the amino terminus of PAK4 and that this binding is not disrupted by co-expression of Nef.

FIG. 7B illustrates the co-precipitation of Rac detected by anti-Rac Western blot, showing that Rac does not strongly bind PAK4.

FIG. 8A shows that over-expression of a truncated GEF protein (containing only the catalytic and pleckstrin homology domains) strongly activates Tat transcriptional activity (as measured using an HIV-LTR transcriptional reporter assay), but in a constitutive, unregulated fashion.

FIG. 8B shows that over-expression of either a longer GEF protein (with a full carboxy-terminal tail) or the PAK4 protein does not stimulate Tat greatly, but when both are co-expressed with Tat they synergize to strongly activate Tat transcriptional activity.

FIG. 9 shows that activation of Tat by PAK4 is strongly inhibited by Rac, and strongly enhanced by Cdc42. Expression of dominant-negative Rac (RacNl7) or even wild-type Rac blocks PAK4 activation of Tat and even basal Tat activity. Wild-type Cdc42 or an activated mutation of Cdc42 (61L) strongly enhance activation of Tat by PAK4, consistent with Cdc42 (but not Rac) being the substrate of GEF guanyl nucleotide exchange factor activity and mediating its effects on Tat.

FIG. 10 shows Nef acetylation by Tip60 in vivo in COS cells transfected with FLAF-Nef and additionally transfected either with or without Tip60. Nef was immunoprecipitated with anti-FLAG followed by Western blot with anti-acetylated lysine antibody (Catalog #9441, Cell Signaling Technology, Inc.). Amount of Nef protein is the same in the two lanes (data not shown).

FIG. 11 is a comparison of the sequence of human (SEQ ID NO:1) and Drosophila PAK4 (SEQ ID NO:2).

### DETAILED DESCRIPTION OF THE INVENTION

The present invention is based, in part, on the identification and sequencing of a novel serine/threonine kinase, termed “PAK4” herein. The invention is also based, in part, on the discovery of HIV-Tat and HIV-NEF complex formation with PAK4, Cdc42-GEF (guanyl-nucleotide exchange factor), and/or the acetyl-transferase Tip60, which complex formations regulate the activity of HIV-Tat, HIV-NEF and/or Tip60, respectively. The identification of these interactions provides a novel means for identifying compounds that modulate the activity of these enzymes, and or for using such compounds to modulate HIV transcriptional enzymatic activity, as further described below. All references cited are hereby incorporated herein by reference.

PAK4 Characterization & Isolated PAK4 DNA

PAK4 was initially identified as an expressed sequence tag in the GenBank database (Genbank accession number T83145, IMAGE clone 110764) during a search for novel members of this interesting kinase family. This expressed sequence tag was then used to screen a human SK-N-MC cell cDNA library in order to isolate a full length cDNA (GenBank accession number AF005046; SEQ ID NO:3 and SEQ ID NO:4), as further described in Example 1.

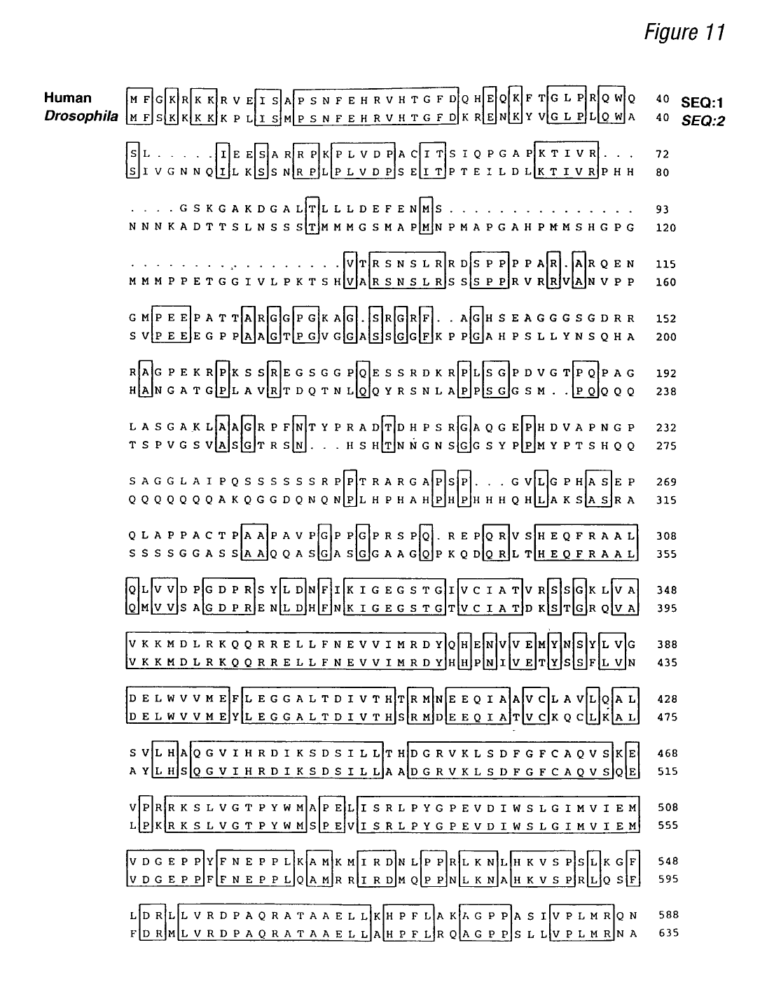

A Drosophila homolog of PAK4 was characterized at the same time by searching the Berkeley Drosophila Genome Database (www.fruitfly.org) with the sequence of the human Pak4 cDNA. A Drosophila cDNA clone (LD05866) that turned out to be full length was obtained from the Berkeley Drosophila Genome Project (GenBank Accession No. AF031517; SEQ ID NO:5 and SEQ ID NO:6; since published by Melzig et al. (1998) Current Biology 8:1223-1226), as further described in Example 1. FIG. 11 compares the sequence of the human and Drosophila Pak4 proteins, highlighting domains of functional significance including the kinase domain and G-protein binding domain.

The invention provides, therefore, in part, isolated DNA molecules encoding the novel PAK4 kinase. In a preferred embodiment, the invention provides an isolated DNA sequence encoding PAK4 serine/threonine kinase, wherein the sequence comprises SEQ ID NO: 1 or conservative mutants or variants thereof. As used herein, “conservative mutants or variants” of a nucleotide or peptide sequence means sequences varying from the specific nucleotide or peptide sequence due to the degeneracy of the genetic code or to point mutations or variances in sequence that due not affect the activity of the encoded protein. In another embodiment, the invention provides a method for producing PAK4 protein comprising the steps of: (a) transfecting a cell with a vector comprising a DNA sequence encoding PAK4, and (b) culturing said cell under conditions suitable for the expression of the desired vector. In another embodiment, the invention provides recombinant PAK4 protein produced by this method, the protein comprising the amino acid sequence of SEQ ID NO: 4 or conservative mutants or variants thereof.

Cdc42-GEF Characterization and Isolated Cdc42-GEF DNA

In order to help characterize the cellular function of the novel PAK4 kinase disclosed herein, a yeast two-hybrid screen of a human brain library was carried out in order to identify potential interacting proteins, as further described in Example II. During this screen, using the human PAK4 open reading frame as bait, a carboxy-terminal fragment of a putative rho-family guanyl nucleotide exchange factor (GEF) previously known only as a cDNA sequence (GenBank Accession No. AB002378; SEQ ID NO:7 and SEQ ID NO:8)) was identified. As described below, Cdc42-GEF complexes with HIV-Tat and modulates its activity.

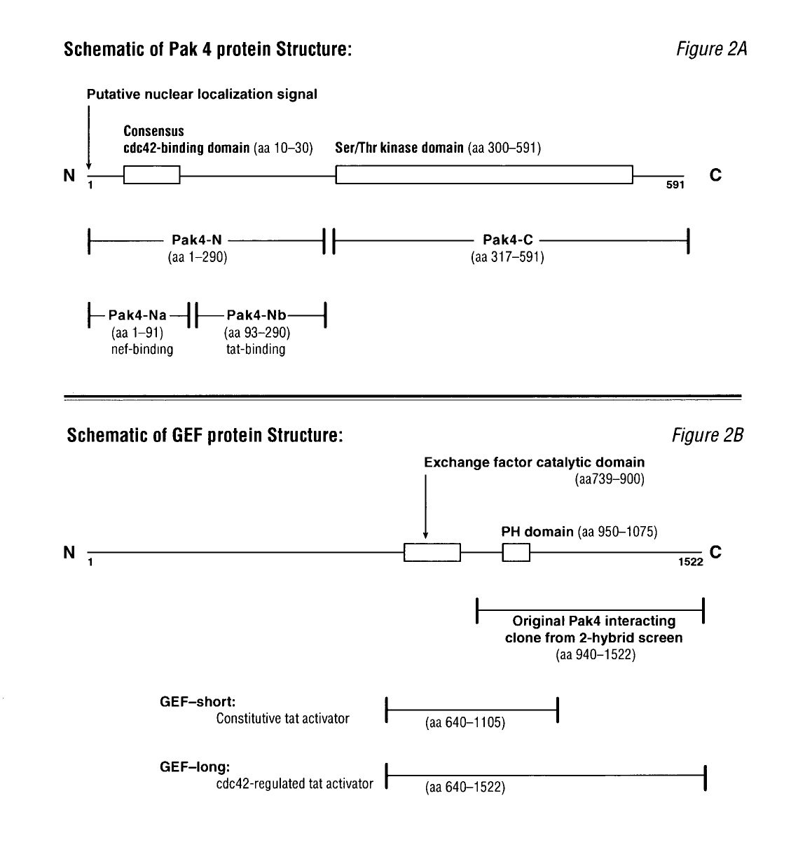

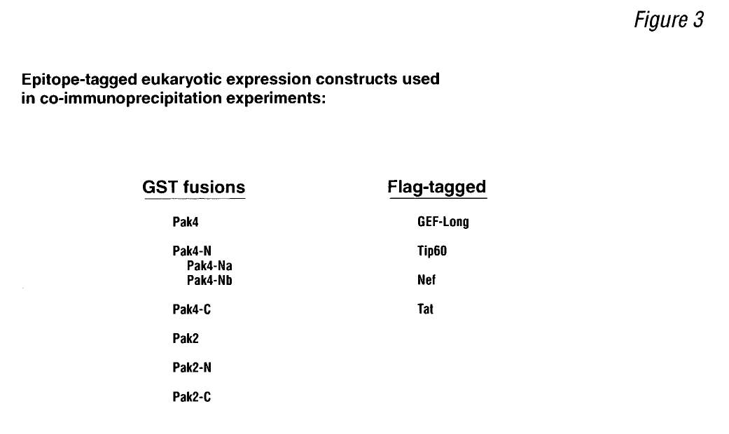

Accordingly, the invention also provides, in part, isolated DNA molecules encoding Cdc42-GEF protein, and expression vectors containing the same. In a preferred embodiment, there is provided a vector for expressing Cdc42-specific GEF (guanyl-nucleotide exchange factor), said vector comprising a DNA sequence selected from the group consisting of SEQ ID NO: 7, residues 640 to 1105 of SEQ ID NO: 7, residues 640 to 1522 of SEQ ID NO: 7, and conservative mutants or variants thereof. As further described in Example II below, the short form (residues 640-1522) contains the putative catalytic domain and pleckstrin homology (PH) domains (FIGS. 2A and 2B), and the long form (residues 640-1522) contains these domains plus the 400 amino acid residues C-terminal of the PH domain. The GEF long form modestly activates Tat, but dramatically activated Tat in combination with PAK4, while GEF short form constitutively and dramatically activates Tat (FIG. 3), as further described in Example 3.

In another embodiment, the invention provides a method for producing Cdc42-GEF protein, the method comprising the steps of: (a) transfecting a cell with a vector comprising a Cdc42-GEF DNA sequence, and (b) culturing said cell under conditions suitable for the expression of the desired vector. The invention also provides a recombinant Cdc42-GEF protein produced by this method, wherein said protein comprises the amino acid sequence of SEQ ID NO: 8 or conservative mutants or variants thereof.

Co-immunoprecipitation Experiments

In order to confirm the PAK4 protein interaction complexes identified in yeast (see Example 1), epitope-tagged eukaryotic expression constructs were made so that protein-protein interactions and complexes could be further tested in vivo by means of co-immunoprecipitation experiments, as further described in Example 2. As part of this screening, given the interaction with Tip60, the interaction of PAK4 with HIV-Tat itself was examined. Further, given the potential HIV connection and mindful of the literature reporting HIV-Nef interacting with a PAK-family kinase (Trono and Wang (1997) Chemistry and Biology 4:13-15), the possible interactions of HIV-Nef with PAK4 was examined. See Example 2.

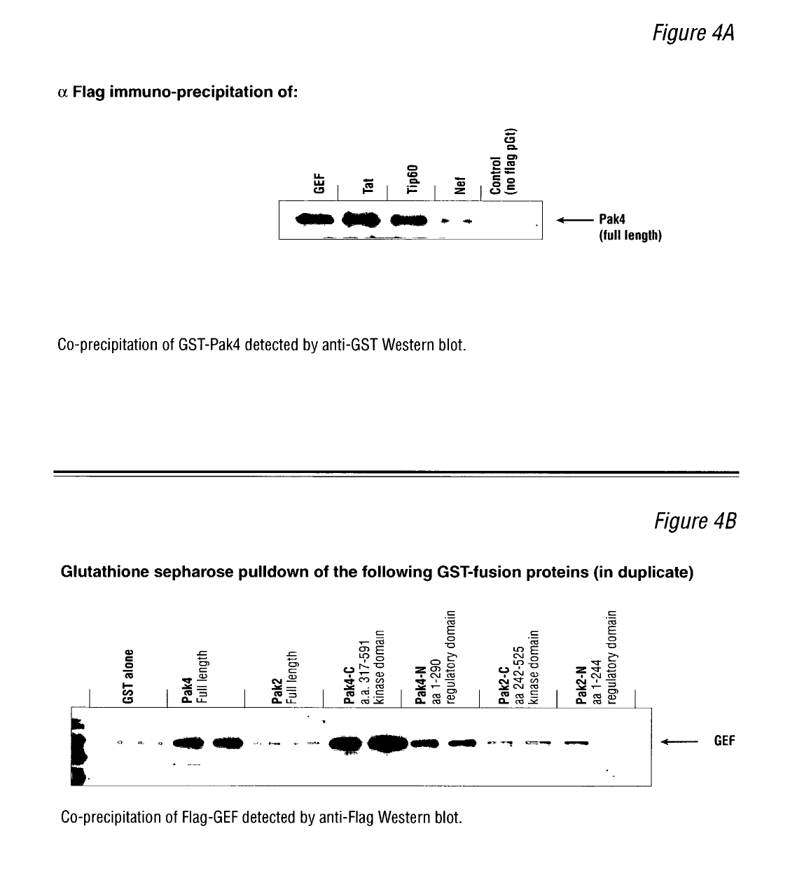

The interactions between PAK4 and the cellular proteins Tip60 and GEF were confirmed by co-immunoprecipitation of the proteins expressed in COS cells (FIG. 4A); see Example 2. The binding of GEF was specific for PAK4 (i.e. GEF was not co-precipitated by PAK2 (FIG. 4B). The interaction of GEF with PAK4 parallels the recent observation of binding of the PIX family of guanyl nucleotide exchange factors to other members of the PAK family (Manser et al. (1998) Mol. Cell 1:183-192).

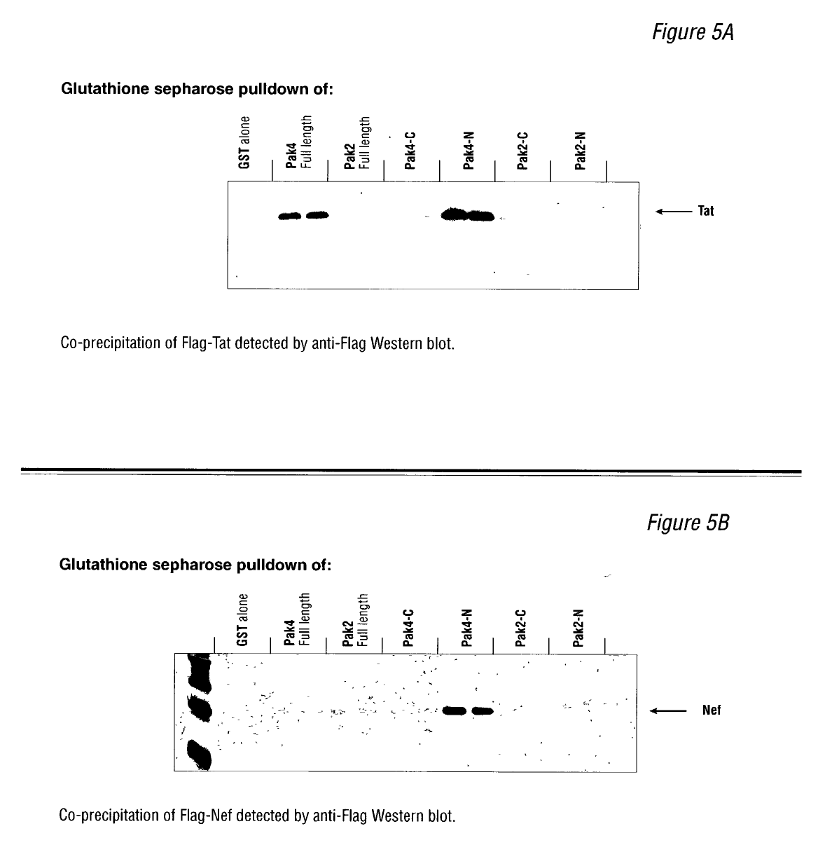

When the possibility of interactions between PAK4 and the HIV accessory proteins Tat and Nef was examined, full-length PAK4 was found to clearly pull down Tat (FIGS. 5A and 5B). In order to localize the Tat-binding region within the PAK4 protein, amino- and carboxy-terminal halves of PAK4 were expressed separately. Co-immunoprecipitation experiments with these PAK4 deletion constructs showed strong binding of both Tat and Nef to the amino-terminal, putative regulatory half of PAK4 (amino acids residues 1-290) but not the carboxy-terminal kinase domain; furthermore this interaction was specific for PAK4, as neither Nef nor Tat bound corresponding PAK2 expression constructs (FIGS. 5A and 5B). See Example 2.

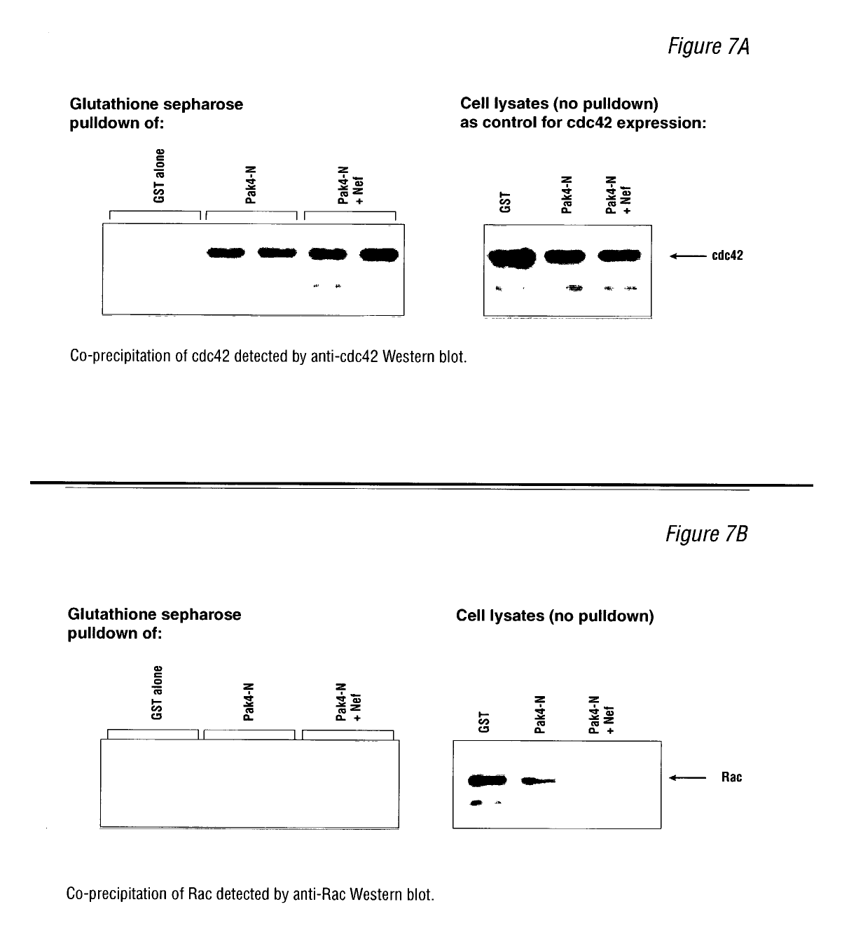

When the specificity of PAK4 for Cdc42 vs. Rac was examined by co-immunoprecipitation of PAK4 co-expressed in COS cells with either of the two small G proteins, it was observed that PAK4 binds Cdc42 not Rac, and furthermore this interaction was not disrupted by co-expressing Nef (FIGS. 7A and 7B).

The regulation of Tat transcriptional activity by PAK4 and PAK4-associated proteins was further examined, as described in Example 3, in COS cells using a reporter construct containing luciferase gene under the control of the HIV-LTR promoter. These experiments indicate that PAK4 and Cdc42-GEF form a protein complex with HIV Tat and modulate its activity.

The regulation of HIV-Nef activity via acetylation by Tip60 and associated binding proteins was further examined, as described in Example 3, in COS cells expressing PAK4, GEF, Tat, and Nef, with or without Tip60. These experiments indicate that HIV-Nef is acetylated in the presence of Tip60. None of the other proteins tested showed acetylation by Tip60, as assayed by Western blot of cell lysates using an antibody that recognizes acetylated lysine regardless of amino acid context.

Modulation of Tat Activity & Screening Methods

The identification of protein-protein interactions and complexes disclosed herein provides a novel means for modulating the activity of HIV transcriptional proteins and screening for compounds that modulate (i.e. activate or inhibit) the activity of these proteins.

Thus, in one embodiment, the invention provides a method for modulating the transcriptional activity of human immunodeficiency virus (HIV) Tat protein, said method comprising modulating the formation of a complex between Tat and at least one modulator complex comprising (i) the serine/threonine kinase PAK4 and the guanyl nucleotide exchange-factor Cdc42-GEF or (ii) PAK4, HIV-NEF, and the acetyl-transferase Tip60. In a preferred embodiment, the modulator complex comprises PAK4/Cdc42-GEF, and the inhibition of formation of a complex between Tat and said modulator complex decreases the transcriptional activity of Tat. In another preferred embodiment, the formation of the complex between Tat and said modulator complex is inhibited by contacting a Tat-expressing cell or cellular preparation with at least one compound that decreases the activity or expression of PAK4 and/or Cdc42-GEF. In another preferred embodiment, the modulator complex comprises PAK4/HIV-NEF/Tip60, and the formation of a complex between Tat and said modulator complex decreases the transcriptional activity of Tat. In still another preferred embodiment, the formation of the complex between Tat and said modulator complex is induced by contacting a Tat-expressing cell or cellular preparation with at least one compound that alters the activity or expression of PAK4 and/or HIV-NEF, and/or Tip60. In a preferred embodiments, the compound is an inhibitor of PAK4 kinase activity or an inhibitor of the GTP exchange factor activity of cdc42-GEF.

The inhibition of a complex between Tat and the PAK4/cdc42-GEF modulator complex decreases the transcriptional activity of Tat, and the inhibition of a complex between Tat and the PAK4/Nef/Tip60 modulator complex increases the transcriptional activity of Tat. As described herein, these characteristics can be used in the search for novel drug candidates against HIV, with the inhibition of the transcriptional activity of Tat as a target. In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target. The cell-free assay involving immunoprecipitation as described below is suitable for a high throughput format that can be designed for robotic automation.

In another embodiment, therefore, the invention provides a method for identifying a compound (e.g. a drug candidate) that inhibits the transcriptional activity of HIV-Tat, said method comprising the steps of: reacting said compound with a complex comprising (i) PAK4/Cdc42-GEF or (ii) HIV-Tat/PAK4/Cdc42-GEF; and determining whether said complex of step (a) is disrupted, wherein said compound is identified as an inhibitor of HIV-Tat transcriptional activity if said complex is disrupted. In preferred embodiments, the complex of step (a) is present in a cellular extract, and the determination of step (b) is accomplished by immunoprecipitation. In another preferred embodiment, the method further comprises the step of (c) confirming that the compound inhibits the in vivo transcriptional activity of Tat by reacting said compound with a cell or cellular preparation comprising a Tat transcriptional reporter. In a preferred embodiment, the transcriptional reporter comprises luciferase activity.

A drug candidate, for example, may be assessed for its capability of inhibiting the transcriptional activity of human immunodeficiency virus (HIV) Tat protein by testing on a cellular extract containing a complex of (i) PAK4/cdc42-GEF or (ii) HIV-Tat/PAK4/cdc42-GEF. The complex is immunoprecipitated with an antibody immobilized, for example, on beads. The immobilized antibody reacts with one of the proteins in the complex, or if one of the proteins is tagged, the antibody also can react against the protein tag. The complex bound to the antibody is released from the antibody after precipitation and washing of the beads by, for example, boiling in SDS-containing buffer. The amount of complex formed in the cellular extract then is determined by analyzing the amount of protein released from the antibody, for example by separating the proteins by SDS-PAGE and probing by Western blot. The influence of a drug candidate on the formation of said complexes is measured against a control incubation that does not contain the drug candidate investigated. The capability of a drug candidate to inhibit the transcriptional activity of human immunodeficiency virus (HIV) Tat protein is measured by its ability to disrupt said complexes.

In another embodiment, the invention provides a method for identifying a compound that inhibits the transcriptional activity of HIV-Tat, the method comprising the steps of: (a) reacting said compound with a mixture comprising (i) PAK4, HIV-NEF, and Tip60 or (ii) HIV-Tat, PAK4, HIV-NEF, and Tip60; and (b) determining whether said compound enhances the formation of a complex comprising (i) PAK4/HIV-NEF/Tip60 or (ii) HIV-Tat/PAK4/HIV-NEF/Tip60, wherein said compound is identified as an inhibitor of HIV-Tat transcriptional activity if the formation of a complex in step(b) is enhanced. In preferred embodiments, the mixture of-step (a) is present in a cellular extract, and the determination of step (b) is accomplished by immunoprecipitation. In another preferred embodiment, the determination of step (b) is accomplished by comparing complex formation to the level of complex formation in a control sample. In still another preferred embodiment, the method further comprises the step of (c) confirming that the compound inhibits the in vivo transcriptional activity of Tat by reacting said compound with a cell or cellular preparation comprising a Tat transcriptional reporter. In a preferred embodiment, the transcriptional reporter comprises luciferase activity.

For example, a drug candidate may be assessed for its capability of inhibiting the transcriptional activity of human immunodeficiency virus (HIV) Tat protein by testing on a cellular extract containing a complex of (i) PAK4/HIV-Nef/Tip60 or (ii) HIV-Tat/PAK4/HIV-Nef/Tip60. The complex is immunoprecipitated with an antibody immobilized, for example, on beads. The immobilized antibody reacts with one of the proteins in the complex, or if one of the proteins is tagged, the antibody also can react against the protein tag. The complex bound to the antibody is released from the antibody after precipitation and washing of the beads by, for example, boiling in SDS-containing buffer. The amount of complex formed in the cellular extract then is determined by analyzing the amount of protein released from the antibody, for example by separating the proteins by SDS-PAGE and probing by Western blot. The influence of a drug candidate on the formation of said complexes is measured against a control incubation that does not contain the drug candidate investigated. The capability of a drug candidate to inhibit the transcriptional activity of human immunodeficiency virus (HIV) Tat protein is measured by its ability to enhance the formation of said complexes.

Other techniques for immobilizing proteins on matrices are also available for use in the subject immunoprecipitation assays. For example, glutathione-S-transferase/PAK4 (GST/PAK4) fusion proteins can be adsorbed onto glutathione sepharose beads (Amersham Pharmacia or Sigma Chemical) or glutathione derivatized microtitre plates, which are then combined with the cdc42-GEF, Nef or Tat polypeptides. In another example, either of the PAK4, cdc42-GEF, Nef or Tat proteins can be immobilized utilizing conjugation of biotin and streptavidin. For instance, biotinylated PAK4 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with PAK4 but which do not interfere with binding of cdc42-GEF, Nef and Tat can be derivatized to the wells of the plate, and the PAK4 trapped in the wells by antibody conjugation. As above, preparations of cdc42-GEF, Nef or Tat polypeptides and a test compound are incubated in the PAK4-presenting wells of the plate, and the amount of complex between PAK4 and and one or more of the above proteins trapped in the well can be quantitated. Exemplary methods for detecting such complexes, in addition to those described above for the GST-immobilized complexes, include immunodetection of complexes using antibodies reactive with the cdc42-GEF, Nef or Tat polypeptides, or which are reactive with the PAK4 protein and compete for binding with the cdc42-GEF, Nef or Tat polypeptides; as well as enzyme-linked assays (see below).

Complex formation between the PAK4 polypeptide and a cdc42-GEF polypeptide, a Tip60 polypeptide, a Nef polypeptide or a Tat polypeptide may be detected by a variety of techniques. In the above outline of the immunoprecipitation technique, an example was given where the proteins are separated by SDS-PAGE and probed by Western blot. Alternatively, modulation of the formation of complexes can be quantitated using, for example, detectably labelled proteins such as radiolabelled (e.g. ³²P, ³⁵S, ¹⁴C or ³H) cdc42-GEF, Nef, or Tat polypeptides. Following incubation, the beads or microtiter plates containing immobilized PAK4 polypeptides are washed to remove any unbound cdc42-GEF, Nef or Tat polypeptides, and the matrix immobilized radiolabel determined directly (e.g. beads placed in scintilant), or in the supernatant after the complexes of PAK4 with cdc42-GEF, Nef or Tat proteins are subsequently dissociated.

In yet another alternative, enzyme-linked assays are used which rely on detecting an enzymatic activity associated with the cdc42-GEF, Nef or Tat polypeptides (instead of the intrinsic activity of the cdc42-GEF, Nef or Tat polypeptides). In the instance of the latter, the enzyme can be chemically conjugated or provided as a fusion protein with cdc42-GEF, Nef or Tat polypeptides. To illustrate, the cdc42-GEF, Nef or Tat polypeptides can be chemically cross-linked or genetically fused with horseradish peroxidase, and the amount of cdc42-GEF, Nef or Tat polypeptides trapped in the complex can be assessed with a chromogenic substrate of the enzyme, e.g. 3,3′-diamino-benzadine terahydrochloride or 4-chloro-1-napthol. Likewise, a fusion protein comprising the cdc42-GEF, Nef or Tat polypeptides and glutathione-S-transferase can be provided, and complex formation quantitated by detecting the GST activity using 1-chloro-2,4-dinitrobenzene (Habig et al (1974) J Biol Chem 249:7130). In yet another application, proteins may be fluorescently labelled (e.g. FITC), and their presence in the complex may then be measured in a fluorometer. Alternatively, the presence of a complex may be assayed by means of fluorescence resonance energy transfer (FRET), whereby one member of the complex is labeled with a fluorescence donor molecule and another labeled with a fluorescence acceptor molecule, such that in the intact complex fluorescence signal is emitted, but on disruption of the complex energy transfer is interrupted and the signal is lost. FRET may be carried out according to protocols well known to those in the art.

In still another embodiment, the invention provides a method for inhibiting the transcriptional activity of HIV-Tat, the method comprising contacting a HIV-Tat-expressing cell with at least one compound selected from the group consisting of:

(i) a compound that decreases activity or expression of PAK4;

(ii) a compound that decreases activity or expression of Cdc42-GEF;

(iii) a compound that increases activity or expression of HIV-NEF; and

(iv) a compound that increases activity or expression of Tip60.

Since the formation of the complexes of (i) PAK4/cdc42-GEF or (ii) HIV-Tat/ PAK4/cdc42-GEF or (iii) PAK4/HIV-Nef/Tip6O or (iv) HIV-Tat/PAK4/HIV-Nef/Tip60, modulate the transcriptional activity of Tat, Tat activity may be regulated by a drug candidate that influences the expression or activity of the proteins involved in the complex. The action on a cell by a drug candidate that decreases the expression of (i) PAK4 and/or (ii) cdc42-GEF or that increases the expression of (i) HIV-Nef and/or (ii) Tip60 will lead to inhibition of the transcriptional activity of Tat as well.

For example, a drug candidate may be added to cells for a certain amount of time before harvesting, a cell extract is made, and the cell extract is tested for the amount of said proteins expressed. The determination of the amount of said proteins is performed, for example, by separating the proteins by SDS-PAGE and probing by Western blot. The influence of a drug candidate on the expression of said proteins is measured against a control incubation that does not contain the drug candidate investigated. The capability of a drug candidate to inhibit the transcriptional activity of human immunodeficiency virus (HIV) Tat protein is measured by its ability to decrease the expression of (i) PAK4 and/or (ii) cdc42-GEF or to increase the expression of (i) HIV-Nef and/or (ii) Tip60. Alternatively, expression may be monitored in a cell-based, high-throughput assay system (e.g. High Content Screening (HCS)), such as HCS sytems offered by Cellomics, Inc.

A drug candidate identified in any of the above assays is further confirmed to inhibit transcriptional activity of Tat in vivo. In this assay, the drug candidate is reacted with a cell or cellular preparation comprising a Tat transcriptional reporter. In a preferred embodiment of this assay, the Tat transcriptional reporter comprises luciferase activity which is measured, for example, using the Roche Biochemicals luciferase assay kit and a Victor2 luminometer (Perkin Elmer Life Sciences).

In addition to the above assays, the present invention further contemplates screening the drug candidates against Tat selected with the above methods in assays that may serve as an intermediate step for clinical trials on humans. Suitable for that purpose, for example, may be a microtiter assay which measures the ability of selected compounds to inhibit HIV-induced cell killing as well as the toxicity of the test compounds to host cells. The basic assay involves infection of CEM-SS cells or other human cells with virus in the presence of the test compound. Quantitation may be performed, for example, spectrophotometrically using the tetrazolium dye MTS (Cell Titer; Promega) which is converted to a soluble, colored formazan product by mitochondrial enzymes present in metabolically active cells at six days post-infection. Confirmatory assays may include, for example, reverse transcriptase, p24 and infectious virus assays, as well as macroscopic and microscopic observation of test wells.

Finally, clinical trials may be performed on drug candidates that have tested positive in all previous biochemical and cell-based assays. The response of human patients to the drug candidates may be monitored by analyzing cells extracted from the patients in the CTL killing assay, the limited dilution analysis (LDA), or by newer methods such as ELISpot and Intracellular cytokine staining (ICC).

Modulation of HIV-NEF and Tip60 Activity & Screening Methods

The identification of HIV-NEF and Tip60 protein complex formation disclosed herein also provides novel methods for the modulation of the activity of either of these enzymes by modulation of such complex formation.

Thus, in one embodiment, the invention further provides a method for modulating the activity of HIV-NEF, the method comprising contacting a HIV-NEF-expressing cell with at least one compound that modulates the acetyl-transferase activity of Tip60. In a preferred embodiment, the compound increases the activity or expression of Tip60. In another preferred embodiment, the compound decreases the activity or expression of Tip60.

In another embodiment, the invention provides a method for identifying a compound that modulates HIV-NEF acetylation by the acetyl-transferase Tip60, the method comprising the steps of (a) reacting said compound with a mixture comprising HIV-NEF and Tip60; and (b) determining whether said compound inhibits or enhances the level of acetylation of HIV-NEF, wherein said compound is identified as a modulator of HIV-NEF acetylation by Tip60 if the level of acetylation in step(b) is inhibited or enhanced.

In still another embodiment, the invention provides a method for identifying a compound that modulates Tip60 acetyl-transferase activity, the method comprising the steps of: (a) reacting said compound with a mixture comprising HIV-NEF and Tip60; and (b) determining whether said compound inhibits or enhances the level of acetylation of HIV-NEF, wherein said compound is identified as a modulator of Tip60 acetyl-transferase activity if the level of acetylation in step(b) is inhibited or enhanced. In a preferred embodiment, the determination of step (b) is accomplished by comparing the level of acetylation of HIV-NEF to the level of acetylation in a control sample. The compound may thus either decrease or increase the activity and/or expression of Tip60. Tip60 acetylates HIV-Nef, and that acetylation in turn may either increase or decrease the activity of HIV-Nef.

As disclosed herein, HIV-Nef has now been shown to be a substrate of the acetyl-transferase activity of Tip60. Thus, for example, the method of the invention may be employed to identify a drug candidate that modulates Tip60 acetyl-transferase activity. In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target. The cell-free assay involving reaction of Tip60 with HIV-Nef as described below is suitable for a high throughput format that can be designed for robotic automation.

In a preferred embodiment, a drug candidate to be screened for its ability to modulate the acetyl-transferase activity of Tip60 is reacted with a mixture comprising HIV-Nef and Tip60. The proteins may be present in a semi-purified or in a purified form. The amount of acetylation of HIV-Nef then is established, for example, by separating the proteins by SDS-PAGE and probing by Western blot with an antibody against acetylated lysine, such as Cell Signaling Technology, Inc. catalog # 9941. The influence of a drug candidate on the acetylation of HIV-Nef, which may be either inhibitory or enhancing, then is established by comparing the level of acetylation of HIV-Nef to the level of acetylation in a control sample.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

Identification of PAK4 and its Binding Partners

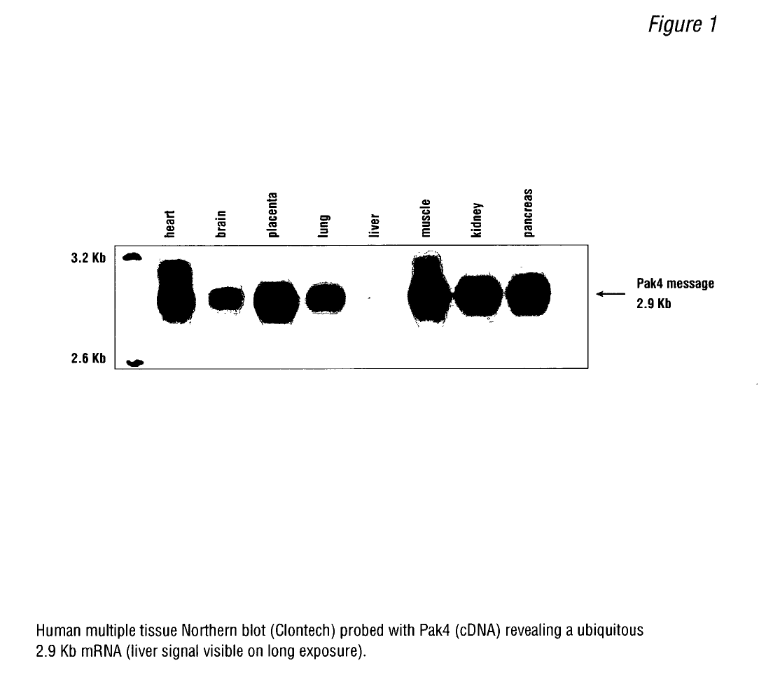

PAK4 kinase was initially found as an expressed sequence tag in the GenBank database (Genbank accession number T83145, IMAGE clone 110764) during a search for novel members of this interesting kinase family. This expressed sequence tag was used to screen a human SK-N-MC cell cDNA library in the vector pCDNA1 (Invitrogen) and isolate a full length cDNA (GenBank accession number AF005046; SEQ ID NO:3 and SEQ ID NO:4). Northern analysis using this cDNA to probe a human multiple tissue northern blot (Clontech) indicates that expression of this gene is ubiquitous (FIG. 1). A BAC human genomic DNA clone (14B11) containing this gene was identified by Research Genetics, Inc. (Huntsville, Ala.), and mapped by them to human chromosome 19q13.

A Drosophila homolog of PAK4 was also characterized at the same time by searching the Berkeley Drosophila Genome Database (www.fruitfly.org) with the sequence of the human Pak4 cDNA. A Drosophila cDNA clone (LD05866) that turned out to be full length was obtained from the Berkeley Drosophila Genome Project (GenBank Accession No. AF031517; SEQ ID NO:5 and SEQ ID NO:6; since published by Melzig et al. (1998) Current Biology 8:1223-1226). FIG. 11 compares the sequence of the human and Drosophila Pak4 proteins, highlighting domains of functional significance including the kinase domain and G-protein binding domain. A Drosophila genomic DNA clone containing this gene was identified using the Drosophila cDNA to screen an array of BAC clones provided by the Berkeley Drosophila Genome Project; these previously mapped clones gave a chromosomal map position for the Drosophila gene of 14E on the X chromosome.

Initial Identification of Interacting Proteins

In order to help characterize the cellular function of this novel kinase, a yeast two-hybrid screen of a human brain library in vector pACT2 was performed to identify potential interacting proteins (Matchmaker GAL4 Two-hybrid system, Clontech). Using the human PAK4 open reading frame cloned into the vector pAS2-1 as bait, a carboxy-terminal fragment of a putative rho-family guanyl nucleotide exchange factor (GEF) previously known only as a cDNA sequence (GenBank Accession No. AB002378; SEQ ID NO:7 and SEQ ID NO:8)) was identified as a complexing protein. Using the amino-terminal half of PAK4 (amino acids 1 to 313, lacking the kinase domain) cloned into the vector pAS2-1 as bait, a putative acetyl-transferase, called Tip60 because it was initially characterized as an HIV-Tat-interacting protein (Kamine et al. (1996) Virology 216:357-366), was identified as a complexing protein. This enzyme is also evolutionarily conserved in Drosophila where it is called Mof and implicated in dosage compensation (Hilfiker et al. (1997) EMBO J. 16(8):2054-2060).

### EXAMPLE 2

In vivo Protein-Protein Binding Interactions of HIV-Tat and -Nef

In order to confirm the PAK4 protein interactions identified in yeast, epitope-tagged eukaryotic expression constructs were made so that protein-protein interactions could be further tested in vivo by means of co-immunoprecipitation experiments. PAK4 and various PAK4 deletions were expressed as GST fusion proteins using a eukaryotic expression vector kindly provided by John Kyriakis, where an amino-terminal GST fusion of the desired protein is expressed under the control of the strong constitutive EF-1α promoter (Mizushima and Nagata (1990) Nucleic Acids Research 18(17):5322). Tip60 and GEF were expressed as FLAG-tagged proteins after cloning into the pFLAG-CMV-2 vector from Sigma-Aldrich (FIGS. 2A, 2B, and 3).

Further, given the interaction with Tip60, the possibility that PAK4 might interact with HIV-Tat itself was tested. The HIV-Tat open reading frame was generated by gene synthesis (Stemmer et al. (1995) Gene 164(1):49-53) using the Tat amino acid sequence from the SF2 isolate of HIV-1 (Genbank accession number AAB59879) converted to DNA sequence using optimal human codon usage tables and the backtranslate program from the Wisconsin Package of the Genetics Computer Group (GCG; Madison, Wis.). The Tat open reading frame was then cloned into the vector pFLAG-CMV-2 in order to express FLAG-tagged protein. Given the potential HIV connection and mindful of the literature reporting HIV-Nef interacting with a PAK-family kinase (Trono and Wang (1997) Chemistry and Biology 4:13-15), a FLAG-tagged Nef (Genbank accession number AAC68849) was also synthesized and expressed in order to investigate a possible interaction with PAK4.

COS cells seeded into 6-well plates were transfected using Fugene-6 (Roche Biochemicals), and various GST- and Flag-tagged proteins expressed or coexpressed for 4248 hrs. Cells were washed with PBS, then 1 ml of the following buffer was added:

20 mM Tris/HCI pH 7.5

50 mM NaCl

1 mM EDTA

1 mM EGTA

0.1% Triton X-100

plus the following phosphatase inhibitors:

1 mM Sodium vanadate

2.5 mM sodium pyrophosphate

1 mM β-Glycerol-phosphate

plus protease inhibitors (Roche Biochemicals Complete cocktail).

Cells were lysed by passing through a 27 gauge needle four times, then spun for 10 minutes at maximum speed in an Eppendorf microcentrifuge. Anti-Flag co-immunoprecipitation was performed by adding 1 μg of antibody to 400 μl of the supernatant from the cell lysate, followed by overnight incubation at 4° C. The next day 20 μl protein A sepharose (or 20 μl of glutathione sepharose in the case of GST-pulldowns) were added, incubated for one hour on a rotator at 4° C., then washed three times with 500 μl buffer. Beads were boiled in SDS-sample buffer, and proteins in the sample analyzed by SDS-PAGE and western blotting.

In the case of pulldowns of GST-fusions of PAK4, PAK4 ΔN, PAK4 ΔC, and PAK2 using glutathione sepharose, the co-precipitation of Flag-tagged protein partners was detected by Western blot with anti-Flag antibody (Kodak). In the case of immunoprecipitation of Flag-tagged gef, tip 60, tat, and nef with M5 anti-Flag antibody and protein A-sepharose, co-precipitated GST-fusion PAK proteins were detected by Western blot with anti-GST antibody.

The interactions (i.e. complexes) between PAK4 and the cellular proteins Tip60 and GEF were confirmed by co-immunoprecipitation of the proteins expressed in COS cells (FIG. 4A). The binding of GEF was specific for PAK4 (i.e. GEF was not co-precipitated by PAK2 (FIG. 4B)), and disrupted by co-expression of Cdc42 or Rac. The interaction of GEF with PAK4 parallels the recent observation of binding of the PIX family of guanyl nucleotide exchange factors to other members of the PAK family (Manser et al. (1998) Mol. Cell 1:183-192).

When the possibility of interactions between PAK4 and the HIV accessory proteins Tat and Nef was examined, full-length PAK4 was found to clearly pull down Tat but not Nef (FIGS. 5A and 5B). In order to localize the Tat-binding region within the PAK4 protein, amino- and carboxy-terminal halves of PAK4 were expressed separately. Co-immunoprecipitation experiments with these PAK4 deletion constructs showed strong binding of both Tat and Nef to the amino-terminal, putative regulatory half of PAK4 (amino acids residues 1-290) but not the carboxy-terminal kinase domain; furthermore this interaction was specific for PAK4, as neither Nef nor Tat bound corresponding PAK2 expression constructs (FIGS. 5A and 5B).

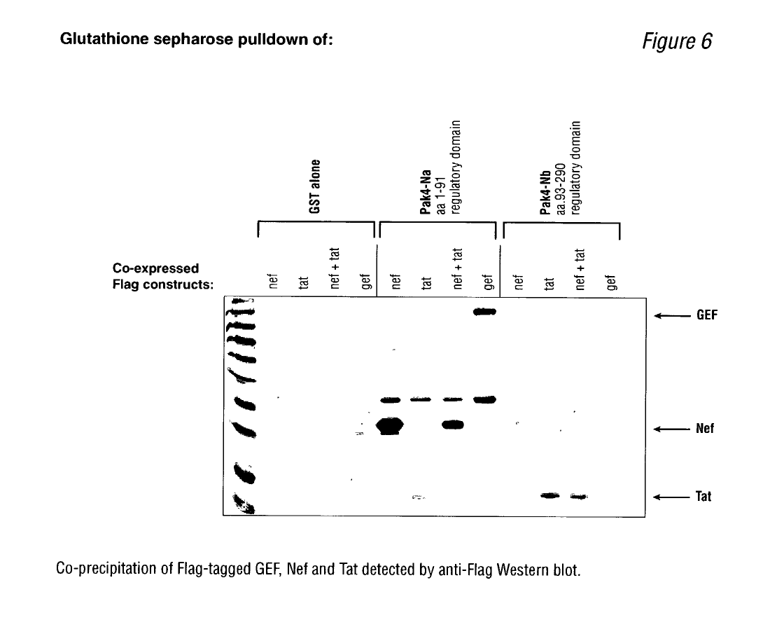

The amino-terminal, putative regulatory half of PAK4 (amino acids residues 1-290) was then further subdivided. PAK4-Na spans residues 1-91, containing a consensus nuclear localization signal and a consensus Rac/Cdc42-binding domain (FIGS. 2A and 2B). PAK4-Nb spans residues 93-290, in between the Rac/Cdc42-binding domain and the kinase domain, a region of no recognizable sequence motifs. This division clearly separated the Nef and Tat binding domains on PAK4, with Nef binding PAK4-Na with its Rac/Cdc42-binding domain, and Tat binding PAK4-Nb (FIG. 6). When the specificity of PAK4 for Cdc42 vs. Rac was examined by co-immunoprecipitation of PAK4 co-expressed in COS cells with either of the two small G proteins, it was observed that PAK4 binds Cdc42 not Rac, and furthermore this interaction was not disrupted by co-expressing Nef (FIGS. 7A and 7B).

### EXAMPLE 3

Regulation of Tat Transcriptional Activity by PAK4 and PAK4-associated Proteins

A Tat transcriptional reporter construct containing luciferase under the control of the HIV-LTR promoter was kindly provided by Ben Berkhout (Verhoef et al. (1997) Nucleic Acids Res. 25(3):496-502). Tat and the HIV-LTR-Luc reporter were co-transfected in COS cells along with PAK4 and PAK4-associated proteins, and luciferase activity assayed in cell lysates 30 hours post-transfection using the Roche Biochemicals luciferase assay kit and a Victor2 luminometer (Perkin Elmer Life Sciences).

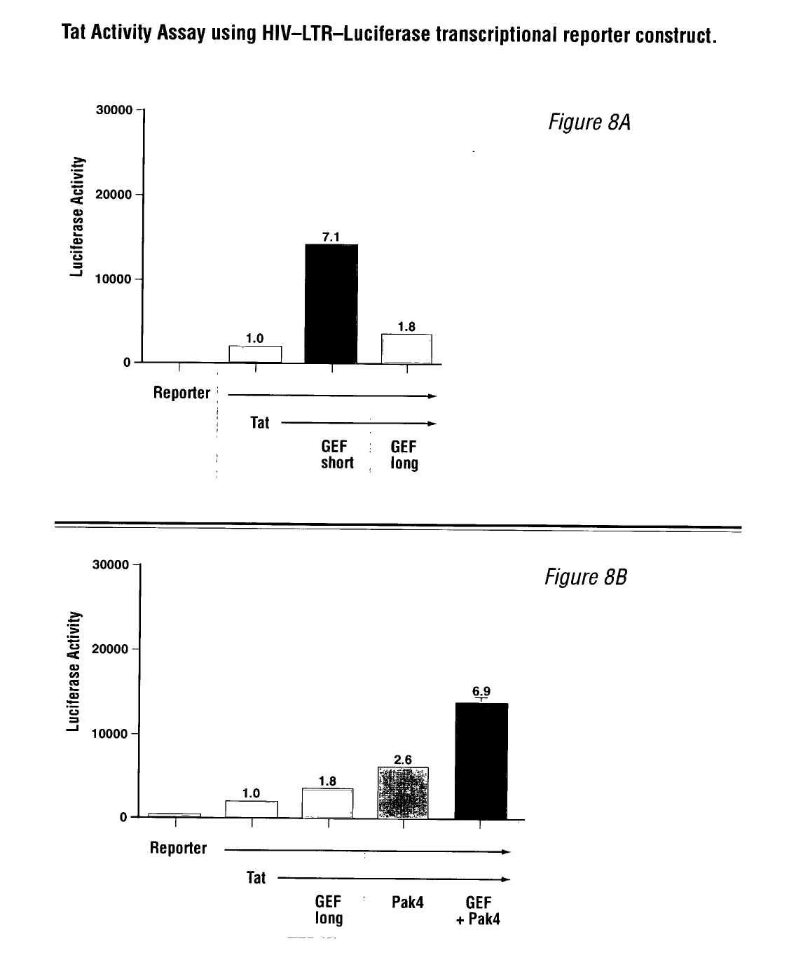

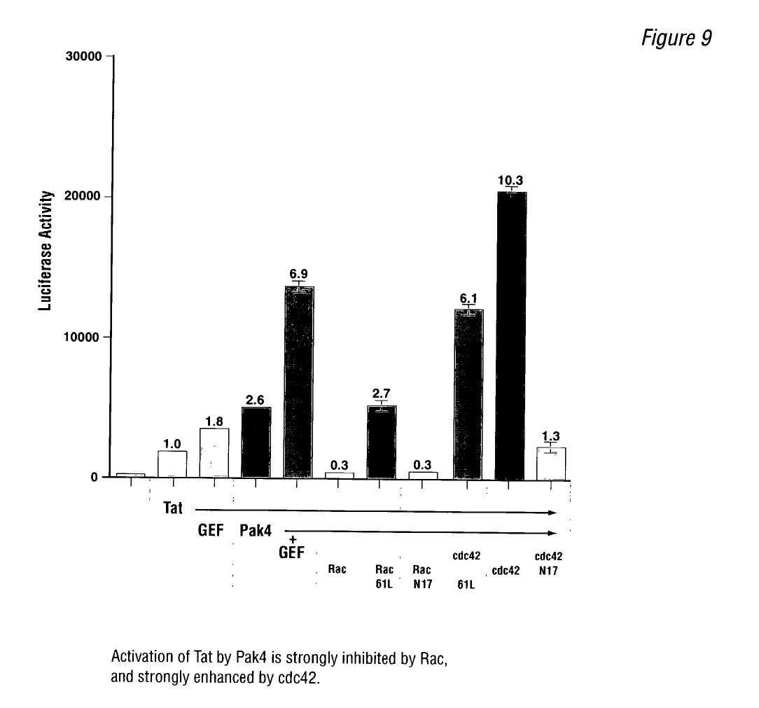

Co-expressing PAK4 with Tat modestly stimulated Tat activity as assayed by the HIV-LTR-Luc transcriptional reporter assay (FIGS. 8A and 8B). Two GEF expression constructs were also tested: a short form (residues 640-1105 of SEQ ID NO: 7) containing the putative catalytic domain and pleckstrin homology (PH) domain (FIGS. 2A and 2B), and a long form (residues 640-1522 of SEQ ID NO: 7) that had these domains plus the 400 amino acid residues C-terminal of the PH domain. The GEF long form modestly activated Tat, but dramatically activated Tat in combination with PAK4. GEF short form constitutively and dramatically activated Tat (FIG. 3). Cdc42 strongly activated Tat, and an activated mutation of Cdc42 (61L) stimulated Tat even more strongly, while Rac consistently inhibited Tat activity (FIG. 9).

Acetylation of HIV-Nef by Tip60

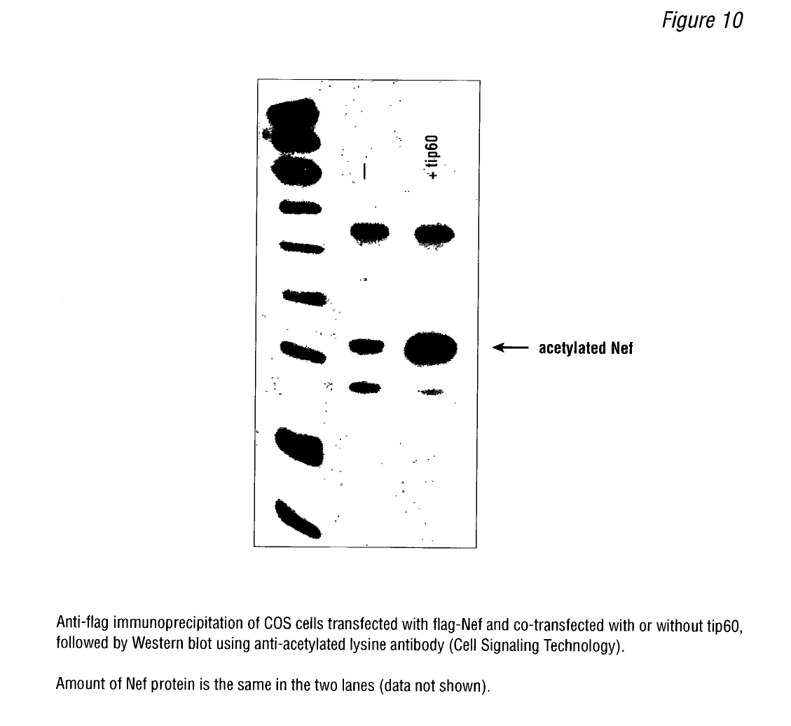

An anti-acetylated lysine antibody from Cell Signaling Technology, Inc. (catalog #9441) was used to look for a potential in vivo substrate of Tip60. PAK4, GEF, Tat and Nef were all expressed in COS cells with or without Tip60, then immunoprecipitated via their respective epitope tags and tested by anti-acetylated lysine western blot for acetylation induced by Tip60 co-expression. HIV-Nef demonstrated a high level of lysine acetylation in the presence of Tip60 (FIG. 10). Tip60 also showed auto-acetylation. None of the other proteins tested showed acetylation (data not shown), at least as detected by this antibody.

What is claimed is:

1. An isolated DNA sequence encoding PAK4 serine/threonine kinase, wherein said sequence comprises SEQ ID NO: 1 or conservative mutants or variants thereof.

2. A vector for expressing Cdc42-specific GEF (guanyl-nucleotide exchange factor), said vector comprising a DNA sequence selected from the group consisting of SEQ ID NO: 7, residues 640 to 1105 of SEQ ID NO: 7, residues 640 to 1522 of SEQ ID NO: 7, and conservative mutants or variants thereof.

3. A method for producing PAK4 or Cdc42-GEF protein, said method comprising the steps of: (a) transfecting a cell with a vector comprising the DNA sequence of claim 1 or with the vector of claim 2, and (b) culturing said cell under conditions suitable for the expression of the desired vector.

4. A recombinant PAK4 protein produced by the method of claim 3, wherein said protein comprises the amino acid sequence of SEQ ID NO: 4 or conservative mutants or variants thereof.

5. A recombinant Cdc42-GEF protein produced by the method of claim 3, wherein said protein comprises the amino acid sequence of SEQ ID NO: 8 or conservative mutants or variants thereof.

6. A method for modulating the transcriptional activity of human immunodeficiency virus (HIV) Tat protein, said method comprising modulating the formation of a complex between Tat and at least one modulator complex comprising (i) the serine/threonine kinase PAK4 and the guanyl nucleotide exchange-factor Cdc42-GEF or (ii) PAK4, HIV-NEF, and the acetyl-transferase Tip60.

7. The method of claim 6, wherein said modulator complex comprises PAK4/Cdc42-GEF, and wherein the inhibition of formation of a complex between Tat and said modulator complex decreases the transcriptional activity of Tat.

8. The method of claim 7, wherein the formation of said complex between Tat and said modulator complex is inhibited by contacting a Tat-expressing cell or cellular preparation with at least one compound that decreases the activity or expression of PAK4 and/or Cdc42-GEF.

9. The method of claim 6, wherein said modulator complex comprises PAK4/HIV-NEF/Tip60, and wherein the formation of a complex between Tat and said modulator complex decreases the transcriptional activity of Tat.

10. The method of claim 9, wherein the formation of said complex between Tat and said modulator complex is induced by contacting a Tat-expressing cell or cellular preparation with at least one compound that alters the activity or express ion of PAK4 and/or HIV-NEF, and/or Tip60.

11. A method for identifying a compound that inhibits the transcriptional activity of HIV-Tat, said method comprising the steps of:
(a) reacting said compound with a complex comprising (i) PAK4/Cdc42-GEF or (ii) HIV-Tat/PAK4/Cdc42-GEF; and
(b) determining whether said complex of step (a) is disrupted, wherein said compound is identified as an inhibitor of HIV-Tat transcriptional activity if said complex is disrupted.

12. The method of claim 11, wherein said complex of step (a) is present in a cellular extract.

13. The method of claim 11, wherein the determination of step (b) is accomplished by immunoprecipitation.

14. The method of claim 11 further comprising the step of (c) confirming that said compound inhibits the in vivo transcriptional activity of Tat by reacting said compound with a cell or cellular preparation comprising a Tat transcriptional reporter.

15. The method of claim 14, wherein said transcriptional reporter comprises luciferase activity.

16. A method for identifying a compound that inhibits the transcriptional activity of HIV-Tat, said method comprising the steps of:
(a) reacting said compound with a mixture comprising (i) PAK4, HIV-NEF, and Tip60 or (ii) HIV-Tat, PAK4, HIV-NEF, and Tip60; and
(b) determining whether said compound enhances the formation of a complex comprising (i) PAK4/HIV-NEF/Tip60 or (ii) HIV-Tat/PAK4/HIV-NEF/Tip60;
wherein said compound is identified as an inhibitor of HIV-Tat transcriptional activity if the formation of a complex in step(b) is enhanced.

17. The method of claim 16, wherein said mixture of step (a) is present in a cellular extract.

18. The method of claim 16, wherein the determination of step (b) is accomplished by immunoprecipitation.

19. The method of claim 16, wherein said determination of step (b) is accomplished by comparing complex formation to the level of complex formation in a control sample.

20. The method of claim 16 further comprising the step of (c) confirming that said compound inhibits the in vivo transcriptional activity of Tat by reacting said compound with a cell or cellular preparation comprising a Tat transcriptional reporter.

21. The method of claim 20, wherein said transcriptional reporter comprises luciferase activity.

22. A method for inhibiting the transcriptional activity of HIV-Tat, said method comprising contacting a HIV-Tat-expressing cell with at least one compound selected from the group consisting of:
(i) a compound that decreases activity or expression of PAK4;
(ii) a compound that decreases activity or expression of Cdc42-GEF;
(iii) a compound that increases activity or expression of HIV-NEF; and
(iv) a compound that increases activity or expression of Tip60.

23. A method for modulating the activity of HIV-NEF, said method comprising contacting a HIV-NEF-expressing cell with at least one compound that modulates the acetyl-transferase activity of Tip60.

24. The method of claim 23, wherein said compound increases the activity or expression of Tip60.

25. The method of claim 23, wherein said compound decreases the activity or expression of Tip60.

26. A method for identifying a compound that modulates HIV-NEF acetylation by the acetyl-transferase Tip60, said method comprising the steps of:
(a) reacting said compound with a mixture comprising HIV-NEF and Tip60; and
(b) determining whether said compound inhibits or enhances the level of acetylation of HIV-NEF,
wherein said compound is identified as a modulator of HIV-NEF acetylation by Tip60 if the level of acetylation in step(b) is inhibited or enhanced.

27. A method for identifying a compound that modulates Tip60 acetyl-transferase activity, said method comprising the steps of:
(a) reacting said compound with a mixture comprising HIV-NEF and Tip60; and
(b) determining whether said compound inhibits or enhances the level of acetylation of HIV-NEF;
wherein said compound is identified as a modulator of Tip60 acetyl-transferase activity if the level of acetylation in step(b) is inhibited or enhanced.

28. The method of claim 27, wherein the determination of step (b) is accomplished by comparing the level of acetylation of HIV-NEF to the level of acetylation in a control sample.


SEQUENCE LISTING (10 sequences)

SEQ ID NO: 1 - protein, 588 aa, Homo sapiens
MFGKRKKRVEISAPSNFEHRVHTGFDQHEQKFTGLPRQWQSLIEESARRPKPLVDPACITSIQPGAPKTIVRGSKGAKDGALTLLLDEFENMSVTRSNSLRRDSPPPPARARQENGMPEEPATTARGGPGKAGSRGRFAGHSEAGGGSGDRRRAGPEKRPKSSREGSGGPQESSRDKRPLSGPDVGTPQPAGLASGAKLAAGRPFNTYPRADTDHPSRGAQGEPHDVAPNGPSAGGLAIPQSSSSSSRPPTRARGAPSPGVLGPHASEPQLAPPACTPAAPAVPGPPGPRSPQREPQRVSHEQFRAALQLVVDPGDPRSYLDNFIKIGEGSTGIVCIATVRSSGKLVAVKKMDLRKQQRRELLFNEVVIMRDYQHENVVEMYNSYLVGDELWVVMEFLEGGALTDIVTHTRMNEEQIAAVCLAVLQALSVLHAQGVIHRDIKSDSILLTHDGRVKLSDFGFCAQVSKEVPRRKSLVGTPYWMAPELISRLPYGPEVDIWSLGIMVIEMVDGEPPYFNEPPLKAMKMIRDNLPPRLKNLHKVSPSLKGFLDRLLVRDPAQRATAAELLKHPFLAKAGPPASIVPLMRQN

SEQ ID NO: 2 - protein, 635 aa, Drosophila melanogaster
MFSKKKKKPLISMPSNFEHRVHTGFDKRENKYVGLPLQWASIVGNNQILKSSNRPLPLVDPSEITPTEILDLKTIVRPHHNNNKADTTSLNSSSTMMMGSMAPMNPMAPGAHPMMSHGPGMMMPPETGGIVLPKTSHVARSNSLRSSSPPRVRRVANVPPSVPEEEGPPAAGTPGVGGASSGGFKPPGAHPSLLYNSQHAHANGATGPLAVRTDQTNLQQYRSNLAPPSGGSMPQQQQTSPVGSVASGTRSNHSHTNNGNSGGSYPPMYPTSHQQQQQQQQQAKQGGDQNQNPLHPHAHPHPHHHQHLAKSASRASSSSGGASSAAQQASGASGGAAGQPKQDQRLTHEQFRAALQMVVSAGDPRENLDHFNKIGEGSTGTVCIATDKSTGRQVAVKKMDLRKQQRRELLFNEVVIMRDYHHPNIVETYSSFLVNDELWVVMEYLEGGALTDIVTHSRMDEEQIATVCKQCLKALAYLHSQGVIHRDIKSDSILLAADGRVKLSDFGFCAQVSQELPKRKSLVGTPYWMSPEVISRLPYGPEVDIWSLGIMVIEMVDGEPPFFNEPPLQAMRRIRDMQPPNLKNAHKVSPRLQSFFDRMLVRDPAQRATAAELLAHPFLRQAGPPSLLVPLMRNA

SEQ ID NO: 3 - DNA, 2838 bp, Homo sapiens
  CDS (211)..(1986)
ccagtgtggtggaattcgtccgcggtggtggcggtgcaagagagctgagggaggcgcgagggcgcggagttccaggtcgagcagttaggccgcgagcgactgcggcgccgagccgatgagtaacccgaagcccctagaggagtggtcacctgcctgagggcacttctgtcccaccagcatcagaccaggccgcaccgagtccccggcaccatgtttgggaagaggaagaagcgggtggagatctccgcgccgtccaacttcgagcaccgcgtgcacacgggcttcgaccagcacgagcagaagttcacggggctgccccgccagtggcagagcctgatcgaggagtcggctcgccggcccaagcccctcgtcgaccccgcctgcatcacctccatccagcccggggcccccaagaccatcgtgcggggcagcaaaggtgccaaagatggggccctcacgctgctgctggacgagtttgagaacatgtcggtgacacgctccaactccctgcggagagacagcccgccgccgcccgcccgtgcccgccaggaaaatgggatgccagaggagccggccaccacggccagagggggcccagggaaggcaggcagccgaggccggttcgccggtcacagcgaggcgggtggcggcagtggtgacaggcgacgggcggggccagagaagaggcccaagtcttccagggagggctcagggggtccccaggagtcctcccgggacaaacgccccctctccgggcctgatgtcggcaccccccagcctgctggtctggccagtggggcgaaactggcagctggccggccctttaacacctacccgagggctgacacggaccacccatcccggggtgcccagggggagcctcatgacgtggcccctaacgggccatcagcggggggcctggccatcccccagtcctcctcctcctcctcccggcctcccacccgagcccgaggtgcccccagccctggagtgctgggaccccacgcctcagagccccagctggcccctccagcctgcacccccgccgcccctgctgttcctgggccccctggcccccgctcaccacagcgggagccacagcgagtatcccatgagcagttccgggctgccctgcagctggtggtggacccaggcgacccccgctcctacctggacaacttcatcaagattggcgagggctccacgggcatcgtgtgcatcgccaccgtgcgcagctcgggcaagctggtggccgtcaagaagatggacctgcgcaagcagcagaggcgcgagctgctcttcaacgaggtggtaatcatgagggactaccagcacgagaatgtggtggagatgtacaacagctacctggtgggggacgagctctgggtggtcatggagttcctggaaggaggcgccctcaccgacatcgtcacccacaccaggatgaacgaggagcagatcgcggccgtgtgccttgcagtgctgcaggccctgtcggtgctccacgcccagggcgtcatccaccgggacatcaagagcgactcgatcctgctgacccatgatggcagggtgaagctgtcagactttgggttctgcgcccaggtgagcaaggaagtgccccgaaggaagtcgctggtcggcacgccctactggatggccccagagctcatctcccgccttccctacgggccagaggtagacatctggtcgctggggataatggtgattgagatggtggacggagagcccccctacttcaacgagccacccctcaaagccatgaagatgattcgggacaacctgccaccccgactgaagaacctgcacaaggtgtcgccatccctgaagggcttcctggaccgcctgctggtgcgagaccctgcccagcgggccacggcagccgagctgctgaagcacccattcctggccaaggcagggccgcctgccagcatcgtgcccctcatgcgccagaaccgcaccagatgaggcccagcgcccttcccctcaaccaaagagccccccgggtcacccccgccccactgaggccagtagggggccaggcctcccactcctcccagcccgggagatgctccgcgtggcaccaccctccttgctgggggtagatgagaccctactactgaactccagttttgatctcgtgacttttagaaaaacacagggactcgtgggagcaagcgaggctcccaggacccccaccctctgggacaggccctcccccatgttcttctgtctccaggaagggcagcggccctcccatcactggaagtctgcagtgggggtcgctgggggtggagagaacactaagaggtgaacatgtatgagtgtgtgcacgcgtgtgagtgtgcatgtgtgtgtgtgtgcaaaggtccagccaccccgtcctccagcccgcaaggggtgtctggcgccttgcctgacacccagccccctctccccctgagccattgtgggggtcgatcatgaatgtccgaagagtggccttttcccgtagccctgcgccccctttctgtggctggatggggagacaggtcagggccccccaccctctccagcccctgcagcaaatgactactgcacctggacagcctcctcttttctagaagtctatttatattgtcattttataacactctagcccctgcccttattgggggacagatggtccctgtcctgcggggtggccctggcagaaccactgcctgaagaaccaggttcctgcccggtcagcgcagccccagcccgcccacccctgcctcgagttagttttacaattaaaacattgtcttgtttaaaaaaaaaaaaaaaaaaaaaaaaaaa

SEQ ID NO: 4 - protein, 591 aa, Homo sapiens
MFGKRKKRVEISAPSNFEHRVHTGFDQHEQKFTGLPRQWQSLIEESARRPKPLVDPACITSIQPGAPKTIVRGSKGAKDGALTLLLDEFENMSVTRSNSLRRDSPPPPARARQENGMPEEPATTARGGPGKAGSRGRFAGHSEAGGGSGDRRRAGPEKRPKSSREGSGGPQESSRDKRPLSGPDVGTPQPAGLASGAKLAAGRPFNTYPRADTDHPSRGAQGEPHDVAPNGPSAGGLAIPQSSSSSSRPPTRARGAPSPGVLGPHASEPQLAPPACTPAAPAVPGPPGPRSPQREPQRVSHEQFRAALQLVVDPGDPRSYLDNFIKIGEGSTGIVCIATVRSSGKLVAVKKMDLRKQQRRELLFNEVVIMRDYQHENVVEMYNSYLVGDELWVVMEFLEGGALTDIVTHTRMNEEQIAAVCLAVLQALSVLHAQGVIHRDIKSDSILLTHDGRVKLSDFGFCAQVSKEVPRRKSLVGTPYWMAPELISRLPYGPEVDIWSLGIMVIEMVDGEPPYFNEPPLKAMKMIRDNLPPRLKNLHKVSPSLKGFLDRLLVRDPAQRATAAELLKHPFLAKAGPPASIVPLMRQNRTR

SEQ ID NO: 5 - DNA, 3351 bp, Drosophila melanogaster
  CDS (493)..(2412)
gcacgagcaccacctctatttctggcatcggcaattatttccctaggtttattatttttttgttaagcgaattgcttgtgcgctcgtgctgtcgccataatgtgtgcaatgtgtgccagccagaggagcaagtggtgaaggtgtatcgcttgtgcgcatctcgatttgtccatccatatccaattgaatcccagtttcccagtcgcgtccgctgctatgcgtgtgtgtgtgcgtgcgtgcccgtgtccctgcatgtgtgtgtgtgagtgagatcgggaagaagttgtgtgctgttgttgttgttgtttctgtcggatttgccgttgccatcgtttgttgtttgtgcattttccattgtcgggcgacgcaacaaaaagaaaaaattacacattaaaaatagagagcaagagcgagagggaaggagtggtgtggagtggaaaagggcggtaaaagggcagaaatttggagcacgggaaatccgagtcgctgaagatgttctcgaagaagaaaaagaaaccgctgatctcgatgcccagcaattttgagcatcgtgtgcacacgggcttcgacaagcgggagaacaaatatgttggcctgcccctccaatgggcgtccattgtgggcaacaatcagatactcaagtcctccaaccgcccgctgccactggtcgatccctcggagattacgcccaccgagattctcgatctgaagactattgtgcgtccgcatcacaacaacaacaaggcggacaccacctcgctcaacagcagcagcacaatgatgatgggctcaatggcgccgatgaatcccatggcacctggcgcacacccaatgatgagccatggccccggaatgatgatgccacccgagacgggcggcatagtcctgccgaagacctctcacgtggccagatccaattcgctgcggagttccagtccgccgcgagtgcgacgggtagccaatgtgccgccatcggtgccggaggaggagggaccaccggcagctggaacaccgggagtaggtggagctagtagcggtggctttaagccacccggtgcccatccctccctgctctataacagtcagcatgcgcacgcgaatggagcaacaggaccactggccgtgcgcacggatcaaaccaacctgcagcagtatcgcagcaatctggccccgccatccggcggctccatgccccagcaacagcagacttcgcccgtgggttcggtggccagtggcacgcgatccaatcactcgcacacgaacaatggcaacagcggcggcagctatcctcccatgtatcccacaagccatcagcagcagcagcagcagcaacagcaggccaaacagggtggcgatcagaaccaaaatcctctgcatccgcatgctcatccgcatccgcaccatcaccaacatttggccaagtcggcgtccagggcatccagttccagcgggggagcgagcagtgctgcccagcaggcgagcggcgccagtggaggagcggcgggtcagccaaagcaggaccaacgactcacccacgaacagttccgtgccgcccttcagatggtggtctccgccggtgatccacgcgagaacctcgaccactttaacaaaataggcgagggctccacgggcaccgtatgcattgccacggacaaatccacaggtcgccaggtggccgtgaagaagatggatctgcgcaaacagcagcgacgggagctgctattcaacgaggtcgtcatcatgcgggactaccatcatcccaatatcgtggagacatactccagctttctggtcaacgatgagctttgggtggtgatggagtacctcgagggcggcgccctcaccgatattgtcacccattcgcgcatggacgaggagcagatagccaccgtctgcaagcaatgcttgaaggctttggcctatttgcactcacagggcgtcattcatcgcgacatcaagtcggactcgattctgctggccgccgatggtcgcgtgaagctatcggactttggattctgcgcccaggtgtcacaggagctgccgaagcgcaagagtctggttggcacgccgtattggatgtcgccggaggtcatatcgcgcctgccgtacggcccggaagtggatatctggtcgctgggcatcatggtcatcgagatggtggacggcgagccgccgttcttcaacgaaccgccgctgcaggcgatgcgtcgcatccgtgacatgcagccgccgaacctgaagaacgcgcacaaggtctcgccgcgcctgcagtctttcttcgaccggatgctggtgcgggatccggcgcagcgtgccaccgccgccgagctgctggcccatcccttcctgcgccaggcaggaccgccgtcgctgctggttccgctgatgcgcaatgcgcgacaccatccgtagttggatgccactggacaacccagacgatccggctgctcgatgaaagcaatcattcccacagacaacggatcccggcccgtgggcatccatctattaaattctagtattaacattcagtttggcagctgtacttaaatccccggagaatcctcgaaacgttgtaaagtcatttaagctccgcacatccatatccatatccatgcccactaacgttccagaagagaacgcaatgtgcctttcacaagcctcgaaatcgaaattgaaatcgcatcctgcaaccgatacggatggagaaacgatatagcatttatctgcccgagaaccacatttttatatatatatatatatatacatatatgagtatttcagcattttgctctctgtctcttgtcaatattgatttgtgtgcgtgtgtgtttgattttttttaaccagcccataaaacattgttcgaatatatacatatatacgctaagagctaatgttaaaaatctatatatatatatatatacacatatattttttataaaaacaaatcctaattctaagcaaaagcgaaacaacgaaggtgtgaatcgaagggagacagaaaacgaaagagaaagagatagcgttaggcggaagagcatatacaatttcgacataaatattttttaaacattttacattttacattggtgtgaaaagtttgtacgctccagacatacacatatgcatatatctaaacacattcccgatttcgaaaatattttagaacttttagcttacattctttgttctgtttttttgatttaacccgtgtaattattaattaatgaataaacttaaatgcgaaaatgttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 639 aa, Drosophila melanogaster
MFSKKKKKPLISMPSNFEHRVHTGFDKRENKYVGLPLQWASIVGNNQILKSSNRPLPLVDPSEITPTEILDLKTIVRPHHNNNKADTTSLNSSSTMMMGSMAPMNPMAPGAHPMMSHGPGMMMPPETGGIVLPKTSHVARSNSLRSSSPPRVRRVANVPPSVPEEEGPPAAGTPGVGGASSGGFKPPGAHPSLLYNSQHAHANGATGPLAVRTDQTNLQQYRSNLAPPSGGSMPQQQQTSPVGSVASGTRSNHSHTNNGNSGGSYPPMYPTSHQQQQQQQQQAKQGGDQNQNPLHPHAHPHPHHHQHLAKSASRASSSSGGASSAAQQASGASGGAAGQPKQDQRLTHEQFRAALQMVVSAGDPRENLDHFNKIGEGSTGTVCIATDKSTGRQVAVKKMDLRKQQRRELLFNEVVIMRDYHHPNIVETYSSFLVNDELWVVMEYLEGGALTDIVTHSRMDEEQIATVCKQCLKALAYLHSQGVIHRDIKSDSILLAADGRVKLSDFGFCAQVSQELPKRKSLVGTPYWMSPEVISRLPYGPEVDIWSLGIMVIEMVDGEPPFFNEPPLQAMRRIRDMQPPNLKNAHKVSPRLQSFFDRMLVRDPAQRATAAELLAHPFLRQAGPPSLLVPLMRNARHHP

SEQ ID NO: 7 - DNA, 5790 bp, Homo sapiens
  CDS (746)..(5314)
aattggctcatttaagaatttcaaaacatttaatgtaaaagcttttttttttttaaggaagtccataaattttggttcccagggttgcactggacttggaaggagtgctgttgtgtacatactattgtatggttttatttattattttactgtacaaatcagccgaaagaatttttccaagtgccatttcggatttattaatccttttttttttcctttcctcaaagatatttgctgttgtcatattaagcattggagactagaaaattactttccccctttgagctagagggtctcttgccaacagaaggacagctgagaaagctggatttaaaggatggttttatctgtactttgcagttaacagtgatattttgaaggcacatttttctgtgattcatttttttttggccatagtgctaaccttgaagagattcgtggctgggtttttggtttctgagaaggtcgtagtttttcctcttttcctttttttttttcttttttcttttcttttctttttttttaaagcgggggaggggaagaggggctgagaaaggaaatcatgttcactggtagaagtagagtggagcatcagttaccagggtcctgagagctggaggagaaaggattctatcttcaagttgggaggccctcctctcaccttgctcaaaaattgcaagcgattcaatcctgatcaagacaccaaagctacaggattctggaaccgtggagacaccgagaaaccatgagtgtaaggttaccccagagtatagacaggttaagtagcctgtcttctctgggagattctgcaccagagcgcaagtccccttcccaccatcgccagccttcggatgcctctgagacaacaggtctcgttcaacgctgtgtcattatccaaaaggaccagcatggcttcggcttcacagtcagtggggatcgcattgttctggtgcagtctgtgcggcctggaggtgcagccatgaaggccggtgtgaaagagggcgaccggatcatcaaagtcaacggcaccatggtgaccaatagctcacacctggaagtggtaaagctgatcaaatctggcgcctatgtcgcactcaccctcctgggctcttcaccttcatccatgggcatctctgggctccagcaggacccatccccagcaggagctccccgaatcacgtcagtgatcccctcaccaccacctcctccacctctaccacctccacaacgcatcacaggacccaaacctctgcaggatcccgaagttcaaaaacatgccacccagatcctcaggaatatgctgaggcaggaagaaaaagaattacaggacatacttccactatatggtgacaccagccagagaccatcagaaggccggctctctctggattcccaggagggggacagtggcttggactctgggacagaacgctttccttccctcagtgagtcattgatgaatcggaactcggtactgtcagaccctgggctagacagtcctcgaacctcccctgtgatcatggccagggtggcccagcaccacaggcggcagggctcggatgcagcagtcccctcaaccggtgaccagggtgtagatcaaagcccaaagcctttaattattggcccagaggaagactatgacccgggttatttcaacaacgagagcgacatcatattccaggatctggagaaactgaagtctcggccagctcacctgggggtttttctacgttacatcttctctcaggcggaccccagtccactgcttttttacctgtgtgcagaagtttatcagcaggcaagccccaaggattcccgaagcttggggaaagacatctggaatattttcctggagaaaaatgcgcctctgagagtgaagatccctgagatgctacaggctgaaattgactcgcgcctgcggaacagcgaagatgcccgtggtgttctctgtgaagctcaagaggcagccatgcctgagatccaagagcagatccacgactacagaacgaagcgcacactggggctgggcagcctgtatggtgaaaatgacctgctggacctggatggggaccctctccgagagcgccaagtggctgagaagcagctggctgcccttggagatattttgtccaagtatgaggaagacaggagcgcccccatggacttcgccctcaatacctacatgagccatgctgggatccgtcttcgagaggcacgaccttccaacacagctgaaaaggcccagtctgctcctgacaaggacaagtggctaccgttcttccctaagaccaagaagagcagcaattccaagaaagaaaaggatgccttggaggacaagaagcgaaaccctatcctcaaatacattgggaagcccaaaagctcttctcaaagcacatttcatattcccttgtcccctgtggaagtcaaaccaggcaatgtgaggaacatcattcagcactttgagaacaaccagcagtatgatgccccagaacctgggacacaacgactctcgaccggaagctttcctgaggacctgctggagagtgacagttcacgctcagagattcgcctgggccgctctgaaagcctcaagggccgggaagagatgaaacggtctcgaaaggcagagaacgtgccccgctctcgcagtgatgttgacatggatgctgctgcggaggctactcgcctgcaccagtcagcctcgtcctctacctccagcctctccaccaggtctcttgagaacccaacccctccattcactcccaaaatgggccgcaggagcattgagtcccccagtttggggttctgcacagataccctccttccccacctcctagaggatgatctgggccagctgtctgacctggagccagagccagatgcccaaaattggcagcatacagtgggcaaggatgtggtggctgggctaacccagcgggagattgaccggcaagaggtcatcaatgagctgtttgtgactgaagcttcccacctgcgcacactccgggtcctggacctgatcttctaccagcgaatgaagaaggagaacctgatgccccgggaggagctggcccggctcttcccgaacctgcctgaactcatagagattcacaattcctggtgtgaagccatgaagaagctccgggaggaaggccccatcatcaaagagatcagtgacctcatgctggcccggtttgatggccctgcccgagaggaactccagcaagtggctgcacagttctgttcctatcagtcaatagccctagagctaatcaagaccaagcaacgcaaggagagtcgattccagctcttcatgcaggaggctgagagccaccctcagtgtcggcggctgcagctgagagacctcatcatctctgagatgcagcggctcaccaagtacccgctgctgctggagagcatcatcaagcacacagagggtggcacctctgagcatgagaagctgtgccgggcccgggaccagtgccgggagattctcaagtatgtgaatgaagcggtaaaacaaacagagaaccgccaccgtttagagggctaccagaaacgcctggatgccaccgccctggagagggccagcaaccccctggcagcagagttcaagagcctggatcttacaaccagaaaaatgatccatgagggacccctgacctggaggatcagcaaggataagaccttggacctccacgtgctgctgctggaggacctcctagtgctgctacagaaacaggatgagaagctattgctgaagtgccacagcaagactgctgtgggctcctcagacagcaagcagaccttcagccccgtgctcaagctcaatgctgtgctcatccgctctgtggccacagataaacgggccttcttcatcatctgcacctccaagctgggcccaccccagatctatgagctggttgcattgacgtcatcagacaagaacacatggatggagctcttagaagaggccgtgcggaatgccaccaggcaccccggagctgccccaatgcccgtccatcctccacccccaggtccccgggagccagcccagcagggccccacacccagcagggtagaactggatgactcagacgtgttccatggtgaacctgaacctgaggagctgcctggaggcactgggtcccagcagagggtccaagggaagcaccaggtcctgctagaggaccctgagcaggagggcagtgcagaggaagaggaactgggtgtcctgccttgcccttccacatccctggatggagagaacaggggcatcaggacaaggaaccccatccacttggccttcccaggccctctgttcatggaagggctcgctgactccgctctggaagatgtggagaacctgcgacatctgatcctgtggagcctgctgccaggtcacaccatggaaactcaggctgcccaggagcccgaggacgacctgacacccacaccttctgtcatcagcgtcacctctcacccctgggacccaggctccccagggcaagcaccccctgggggtgaaggggacaacacccagcttgcagggctggagggggaacggccagagcaggaagacatgggtctctgttctctggaacacctacccccaaggaccagaaattctgggatatgggagtctccagaactggacaggaatctggctgaagatgcttcaagcacagaggcagcaggaggttacaaagttgtgagaaaagctgaggtggcaggcagcaaggttgtccctgcactaccagagagtggccagtcagagcctgggccacctgaagtggaaggcggaacaaaggctacggggaactgcttttatgtcagcatgccatcaggacccccggactcaagcaccgaccactcagaggcacccatgagcccccctcagcctgacagcctccctgcagggcagacagagcctcagcctcagctgcagggaggcaacgatgatccaagacgccccagccgctctcctccaagcctggccctcagggacgtgggcatgatcttccataccattgagcagctcactctcaagctcaacaggctcaaggatatggagctggcccacagagagctgctcaagtcccttgggggagagtcatctggtggcaccacgcctgtgggcagtttccacacagaagcagctagatggacagatggctccctctcacctcccgctaaggagcccctagcttctgactccaggaacagccatgaactggggccctgccctgaggatggctctgacgcccccctggaagacagcacagcagacgcagccgcgtcaccaggaccataaccgtacaaaccaccaaatcctctgcgtccccactcctccttcagggactggcctgagaccggggcacagggtaggggggatcccaacactcctccctgtggaggaggcagttagggaaactaggatccagccaaggcccggggggagacccgcatgttgcttggtctgctcaagtcggagtcaggtttcagtgtcttttccctcccttagcccaaccctccaaggcctcatgtctcctaagcatgctgactgcatccgaaaggcccccactcaccatggtctgccctcaccccacatatgtgtgtacacgcgcacgcctgtatgtgcgctgccctcagacatgcaagtgaaaggaggaggcttctgtgtaaatgcactttcttcctcccctctttctccataagaccccaggcagaggtgggtgcctcccctcccctctttgtcactttggtttcctataaatatgtatgtatcgt

SEQ ID NO: 8 - protein, 1522 aa, Homo sapiens
MSVRLPQSIDRLSSLSSLGDSAPERKSPSHHRQPSDASETTGLVQRCVIIQKDQHGFGFTVSGDRIVLVQSVRPGGAAMKAGVKEGDRIIKVNGTMVTNSSHLEVVKLIKSGAYVALTLLGSSPSSMGISGLQQDPSPAGAPRITSVIPSPPPPPPLPPPQRITGPKPLQDPEVQKHATQILRNMLRQEEKELQDILPLYGDTSQRPSEGRLSLDSQEGDSGLDSGTERFPSLSESLMNRNSVLSDPGLDSPRTSPVIMARVAQHHRRQGSDAAVPSTGDQGVDQSPKPLIIGPEEDYDPGYFNNESDIIFQDLEKLKSRPAHLGVFLRYIFSQADPSPLLFYLCAEVYQQASPKDSRSLGKDIWNIFLEKNAPLRVKIPEMLQAEIDSRLRNSEDARGVLCEAQEAAMPEIQEQIHDYRTKRTLGLGSLYGENDLLDLDGDPLRERQVAEKQLAALGDILSKYEEDRSAPMDFALNTYMSHAGIRLREARPSNTAEKAQSAPDKDKWLPFFPKTKKSSNSKKEKDALEDKKRNPILKYIGKPKSSSQSTFHIPLSPVEVKPGNVRNIIQHFENNQQYDAPEPGTQRLSTGSFPEDLLESDSSRSEIRLGRSESLKGREEMKRSRKAENVPRSRSDVDMDAAAEATRLHQSASSSTSSLSTRSLENPTPPFTPKMGRRSIESPSLGFCTDTLLPHLLEDDLGQLSDLEPEPDAQNWQHTVGKDVVAGLTQREIDRQEVINELFVTEASHLRTLRVLDLIFYQRMKKENLMPREELARLFPNLPELIEIHNSWCEAMKKLREEGPIIKEISDLMLARFDGPAREELQQVAAQFCSYQSIALELIKTKQRKESRFQLFMQEAESHPQCRRLQLRDLIISEMQRLTKYPLLLESIIKHTEGGTSEHEKLCRARDQCREILKYVNEAVKQTENRHRLEGYQKRLDATALERASNPLAAEFKSLDLTTRKMIHEGPLTWRISKDKTLDLHVLLLEDLLVLLQKQDEKLLLKCHSKTAVGSSDSKQTFSPVLKLNAVLIRSVATDKRAFFIICTSKLGPPQIYELVALTSSDKNTWMELLEEAVRNATRHPGAAPMPVHPPPPGPREPAQQGPTPSRVELDDSDVFHGEPEPEELPGGTGSQQRVQGKHQVLLEDPEQEGSAEEEELGVLPCPSTSLDGENRGIRTRNPIHLAFPGPLFMEGLADSALEDVENLRHLILWSLLPGHTMETQAAQEPEDDLTPTPSVISVTSHPWDPGSPGQAPPGGEGDNTQLAGLEGERPEQEDMGLCSLEHLPPRTRNSGIWESPELDRNLAEDASSTEAAGGYKVVRKAEVAGSKVVPALPESGQSEPGPPEVEGGTKATGNCFYVSMPSGPPDSSTDHSEAPMSPPQPDSLPAGQTEPQPQLQGGNDDPRRPSRSPPSLALRDVGMIFHTIEQLTLKLNRLKDMELAHRELLKSLGGESSGGTTPVGSFHTEAARWTDGSLSPPAKEPLASDSRNSHELGPCPEDGSDAPLEDSTADAAASPGP

SEQ ID NO: 9 - DNA, 2215 bp, Homo sapiens
  CDS (242)..(1783)
actcagtagaccgccactggctgtgcacgttatggggtttccacctagggctcggcctgaggcttgtaacactccgttttcccccgagtcacaggggcagtcttgcccctcgcagctgggtcgcggtgtctctcaaaggtccccctctacaggggcttcgtgaggcccgggcccacagggcgctcggtcccggaagtgacgtctcccagaggggccggaagtggcagtggagggagggaagatggcggaggtgggggagataatcgagggctgccgcctacccgtgctgcggcggaaccaggacaacgaagatgagtggcccctggccgagatcctgagcgtgaaggacatcagtggccggaagcttttctacgtccattacattgacttcaacaaacgtctggatgaatgggtgacgcatgagcggctggacctaaagaagatccagttccccaagaaagaggccaagacccccactaagaacggacttcctgggtcccgtcctggctctccagagagagaggtgccggcctcggcgcaggccagcgggaagaccttgccaatcccggtccagatcacactccgcttcaacctgcccaaggagcgggaggccattcccggtggcgagcctgaccagccgctctcctccagctcctgcctgcagcccaaccaccgctcaacgaaacggaaggtggaggtggtttcaccagcaactccagtgcccagcgagacagccccggcctcggtttttccccagaatggagccgcccgtagggcagtggcagcccagccaggacggaagcgaaaatcgaattgtttgggcactgatgaggactcccaggacagctctgatggaataccgtcagcaccacgcatgactggcagcctggtgtctgatcgaagccacgacgacatcgtcacccggatgaagaacattgagtgcattgagctgggccggcaccgcctcaagccgtggtacttctccccgtacccacaggaactcaccacattgcctgtcctctacctgtgcgagttctgcctcaagtacggccgtagtctcaagtgtcttcagcgtcatttgaccaagtgtgacctacgacatcctccaggcaatgagatttaccgcaagggcaccatctccttctttgagattgatggacgtaagaacaagagttattcccagaacctgtgtcttttggccaagtgtttccttgaccataagacactgtactatgacacagaccctttcctcttctacgtcatgacagagtatgactgtaagggcttccacatcgtgggctacttctccaaggagaaagaatcaacggaagactacaatgtggcctgcatcctaaccctgcctccctaccagcgccggggctaccggaagctgctgatcgagttcagctatgaactctccaaagtggaagggaaaacagggacccctgagaagcccctctcagaccttggcctcctatcctatcgaagctactggtcccagaccatcctggagatcctgatggggctgaagtcggagagcggggagaggccacagatcaccatcaatgagattagtgaaatcaccagcatcaagaaggaggatgtcatctccactctgcagtacctcaatctcatcaactactacaagggccagtacatcctcacactgtcagaggacatcgtggatggccatgagcgggccatgctcaagcggctcctgcggatcgactccaagtgtctgcacttcactcccaaggactggagcaagagggggaagtggtgaccagacactgcccactgcagtgccaagacggcagcaggactggggctgatagcccaccccgcccccactgcagctcccacaaagcactctaagggagatggggctgaggacagctcaaaaaggagaggacaggcctgcagggcccacttgcccagcaccaaggcgagctccgggctcagaccaactccaaggtcagctggccacagcccaggcctcctctgaagcagggaccagagggagccaggcagctgtgtacagtgagaagggatccggatgggggagctctgtacagagggctggtgattgtaaaaatttcttttgtaaagtagaagttgggggtggggtgggtgctggctgcaaaaatttctggcttctcttacccctattgcccccggcaataaattgtttctatatgccaaaaaaaaaaaaaaa

SEQ ID NO: 10 - protein, 513 aa, Homo sapiens [sequence omitted]